(19) United States
(12) Patent Application Publication
(10) Pub. No.: US 2024/0053369 A1
(43) Pub. Date: Feb. 15, 2024

(54) Methods and Systems for Quantifying Lipoprotein(A) Using Candidate References

(71) Applicants: University of Washington, Seattle, WA (US); The University of Western Ontario, London (CA)

(72) Inventors: Tomas Vaisar, Seattle, WA (US); Santica M. Marcovina, Seattle, WA (US); Marlys Laverne Koschinsky, London (CA); Michael Busiku Boffa, London (CA)

(21) Appl. No.: 18/257,719

(22) PCT Filed: Dec. 16, 2021
(86) PCT No.: PCT/US2021/063923

§ 371 Date: Jun. 15, 2023

Related U.S. Application Data
(60) Provisional application No. 63/127,866, filed on Dec. 18, 2020.

(51) Int. Cl. G01N 33/92; G01N 33/68

(52) U.S. Cl. CPC G01N 33/92; G01N 33/6851; G01N 33/6893; G01N 2800/32

(57) ABSTRACT

The present disclosure provides methods, systems, and kits for quantifying lipoprotein(a) based on measuring selected proteotypic peptide(s). The methods of the disclosure are also useful in determining if a subject is at risk to develop cardiovascular disease. The disclosure also provides methods of determining a reference amount of selected proteotypic peptide(s) in a reference sample.

### CROSS-REFERENCE TO RELATED APPLICATIONS

This application is a national stage application under 35 U.S.C. § 371 of International Application No. PCT/US2021/063923, filed Dec. 16, 2021, which claims the benefit of priority of U.S. Provisional Patent Application No. 63/127,866, filed Dec. 18, 2020, each of which is incorporated herein by reference in its entirety.

### STATEMENT OF FEDERALLY SPONSORED RESEARCH AND DEVELOPMENT

This invention was made with government support under Grant No. R01 HL144558, awarded by the National Institutes of Health. The government has certain rights in the invention.

### SEQUENCE LISTING STATEMENT

The instant application contains a Sequence Listing which has been submitted electronically in ASCII format and is hereby incorporated by reference in its entirety. The Sequence Listing is contained in the text file created on Jun. 8, 2023, having the file name “21-1340-WO-SequenceListing.txt” and is 20,480 bytes in size.

### BACKGROUND OF THE DISCLOSURE

With multiple studies confirming the causal link between lipoprotein(a) (Lp(a)) concentrations and increased risk of cardiovascular disease, this past decade has seen a strongly renewed interest in Lp(a). Lp(a) is a lipoprotein composed of apolipoprotein(a) (apo(a)) covalently bound to a low-density lipoprotein (LDL)-like particle containing one molecule of apolipoprotein B-100. Apo(a) is a complex protein sharing a high sequence homology with several regions of plasminogen, including the protease domain, and the so-called kringle IV (MV) and V domains. The MV domain of apo(a) is formed by ten distinct MV types numbered from 1 to 10. All MV types, except MV type 2 (MV-2), are present as a single copy, while the MV-2 repeats vary from 3 to >40 copies, resulting in a large heterogeneity in apo(a) isoform size circulating in plasma. Apo(a) concentration is generally inversely correlated with apo(a) size and varies widely between individuals.

The unique structural characteristics and size heterogeneity of apo(a) can have strong influence on the accuracy of immunochemical methods used for Lp(a) measurements. Antibodies binding to epitopes in the KIV-2 region will generate multiple antibody-antigen complexes dependent on the size of apo(a). As a consequence, when apo(a) size in the calibrator is smaller or larger than that in the measured sample, Lp(a) concentration will be under or overestimated depending on the method.

Improving method comparability requires a strategy for assay standardization based on the development of a reference method calibrated with a high-purity primary reference material, and commutable secondary reference materials with established traceability. Efforts in standardization of Lp(a) measurement were initiated two decades ago by the International Federation of Clinical Chemistry and Laboratory Medicine (IFCC). A secondary reference material, the WHO/IFCC SRM-2B, was produced in 2000 and its implementation significantly improved between-assay comparability.

The concentration of Lp(a) in SRM-2B was assigned by an enzyme-linked immunosorbent assay (ELISA) method using a purified Lp(a) with value assigned by amino acid analysis as primary calibrator. This ELISA involves a monoclonal antibody directed to a unique epitope located in MV type 9, with no cross-reactivity to the MV-2 region of apo(a), rendering this method insensitive to apo(a) size variability. For this reason, it is considered the gold standard for Lp(a) quantification. Although this ELISA exhibits excellent performance, its accuracy, like that of all methods based on antigen-antibody reactions, may be affected by protein conformational diversity. Therefore, there remains a need for an efficient and accurate method to quantify Lp(a) independently of the size of apo(a) in the samples.

### SUMMARY OF THE DISCLOSURE

Recent reviews and guidelines have identified the lack of standardization as a major hindrance to the broader use of Lp(a) for cardiovascular disease risk assessment. With its ability to accurately quantify proteins in complex matrices, targeted liquid chromatography tandem mass-spectrometry (LC-MS/MS) has become a method of choice for the standardization of biomarkers in clinical practice. Like the ELISA gold standard method that was made independent of apo(a) size polymorphism by selecting a monoclonal antibody not interacting with epitopes in MV-2, LC-MS/MS can be rendered independent of apo(a) size polymorphism by selecting specific quantification peptides uniquely present in the apo(a) sequence and specifically not present in the KIV-2 region that contains a variable number of copies of KIV-2. In addition, LC-MS/MS can be traceable to SI units through a calibration strategy based on a combination of isotope dilution and a calibrator with accurately assigned value of apo(a) concentration. These are recognized key requirements of a reference method for assay standardization.

The inventors developed an accurate method for the quantification of Lp(a) in biological samples (such as plasma) using a targeted LC-MS/MS approach with a double isotope dilution external calibration strategy.

Thus, one aspect of the disclosure provides a method for quantifying apolipoprotein(a) in a biological sample. Such method includes measuring an ion signal of at least two candidate peptides and an ion signal of their stable isotope labeled peptides in the biological sample by mass spectrometry analysis, normalizing the ion signal of at least two candidate peptides with the ion signal of stable isotope labeled peptides to obtain a measured average ion signal of the at least two peptides; and quantifying apolipoprotein(a) using the average ion signal of the at least two peptides and a reference amount of a calibrator.

In certain embodiments, the candidate peptides are selected from TPENYPNAGLTR (SEQ ID NO: 1) (TPEN), TPAYYPNAGLIK (SEQ ID NO: 2) (TPAY), and GISSTTVTGR (SEQ ID NO: 3) (GISS).

One of skill in the art would recognize that measuring apo(a) is a surrogate term for measuring Lp(a). Thus, the amount of apo(a) determined by the methods of the disclosure may be used to quantify the amount of Lp(a) in the biological sample. In addition, the one of skill in the art would recognize that the candidate peptides of the disclosure, such as TPEN, TPAY, and GISS, have been selected to quantify apo(a) in the methods of the disclosure due to their favorable properties suitable for quantification, such as resistance to degradation or ex vivo modification, in contrast to other peptides such as TTEY that do not have properties suitable for quantification of apo(a).

The methods of the disclosure can also be used to determine if a subject is at risk to develop cardiovascular disease. Such method includes measuring an ion signal of at least two candidate peptides together with ion signal of their respective stable isotope labeled peptides in a biological sample; normalizing the ion signal of at least two candidate peptides with the ion signal of stable isotope labeled peptides to obtain an average ion signal of the at least two peptides; quantifying apolipoprotein(a) using the average ion signal of the at least two peptides and a reference amount of a calibrator; and comparing the amount of apolipoprotein(a) to a predetermined amount of lipoprotein(a), wherein a difference in the amount of apolipoprotein(a) in the biological sample and the predetermined amount of lipoprotein(a) is indicative of the risk to develop cardiovascular disease in the subject.

Another aspect of the disclosure provides kits useful for carrying out the methods of the disclosure as described herein. For example, such kit includes (i) two or more stable isotope labeled candidate peptides, and optionally (ii) one or more calibrators. In certain embodiments, such candidate peptides are selected from TPEN, TPAY, and GISS.

Another aspect of the disclosure provides methods of determining a reference amount of apo(a) based on measurement of the average ion signal of at least two peptides in a reference sample. Such methods include:
calibrating the reference sample with a primary reference material comprising the high-purity human recombinant apolipoprotein(a);
identifying two or more peptides for quantification based on a group of factors to obtain the two or more peptides; and
determining the reference average ion signal of the two or more peptides normalized to ion signal of their respective stable isotope labeled peptides based on the amount of the primary reference material.

Another aspect of the disclosure provides a non-transitory computer-readable medium having computer-executable instructions stored thereon that, if executed by one or more processors of a computing device, cause the computing device to perform the normalizing of an ion signal of two or more peptides as described herein.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The accompanying drawings are included to provide a further understanding of the compositions and methods of the disclosure and are incorporated in and constitute a part of this specification. The drawings illustrate one or more embodiment(s) of the disclosure and, together with the description, serve to explain the principles and operation of the disclosure.

FIG. 1 illustrates development and validation of a reference method for the quantification of apo(a) in plasma, according to an embodiment of the disclosure. (A) an outline of the method development and validation, according to an embodiment of the disclosure. (B) a quantification strategy by targeted double isotope dilution LC-MS/MS outlining calibrators and sample preparation, according to an embodiment of the disclosure. (C) a sequence of the human recombinant apo(a) calibrator. Candidate quantification peptides and their locations are highlighted. Red, final quantification peptides; blue, candidate peptides not meeting quantification requirements, according to an embodiment of the disclosure.

FIG. 2 illustrates parallelism assessment of the recombinant r14K calibrator as plots of the linear regression curves of the ratio of the endogenous to Stable Isotope Labeled (SIL) peptide peak areas and the concentration for the 3 final quantification peptides and the 3-peptide mean. Diamond, the r14K calibration; circle, the standard addition of r14K to a sample; square, the serial sample dilution, according to an embodiment of the disclosure.

FIG. 3 illustrates comparison of LC-MS/MS and ELISA methods, according to an embodiment of the disclosure. (A) a Weighted Deming regression: [LC-MS/MS]=3.18(1.08−5.28)+[ELISA]*0.98(0.94−1.02). Red dashed line, regression curve; solid black line, equivalence; blue lines, 95% confidence interval (CI), according to an embodiment of the disclosure. (B) a Bland-Altman plot of relative difference between LC-MS/MS and ELISA measurements. Short dashed lines, 95% CI; long dashed lines, the recommended acceptable optimal bias limits, according to an embodiment of the disclosure. (C) a Plot of relative difference between the LC-MS/MS and ELISA measurement vs. the size of dominant apo(a) isoform expressed in number of MV (y=0.13×0.4, r²=0.003, N.S., N=64), according to an embodiment of the disclosure.

FIG. 4 illustrates amino acid sequence of the human recombinant apolipoprotein(a) containing 14 kringles IV (r14K) used for the calibration of the LC MS/MS method. The kringle IV type 2 (MV-2) domain repeats are highlighted in grey. The final three peptides used for quantification: highlighted; the candidate quantification peptides that did not meet the performance requirements set for the method: dark gray, according to an embodiment of the disclosure.

FIG. 5 illustrates densitometric profile of the electrophoretic separation of 10Ong of pure recombinant 14K apo(a) on a 1.5% agarose gel. Bands were detected by Western blot using a monoclonal antibody according to the protocol published by Marcovina et al. for the measurement of apo(a) isoform sizes (Curr Opin Lipidol 2004, 15(2):167-74), according to an embodiment of the disclosure.

FIG. 6 illustrates evaluation of the purity of the r14K recombinant apolipoprotein(a) by three complementary methods: (A) Electrospray Differential Mobility Analysis (ESI-DMA), (B) SDS-PAGE on a 3-8% Tris-Acetate gel stained with Coomassie Blue, and (C) MiniQ anion-exchange FPLC with UV absorption detection at 280 nm, according to an embodiment of the disclosure.

FIG. 7 illustrates digestion time course for all candidate quantification peptides, according to an embodiment of the disclosure. Ratios of the peak areas of endogenous to SIL peptides as a function of the digestion time for (A) TPAY, (B) TPEN, (C) GISS, (D) GSFS, (E) LFLE and (F) TTEY (Note: Not all ratios at 12 and 18 h could be calculated for TTEY because some peak areas were undetectable). For each time-point, 3 independent digests were injected in duplicate (n=6). Error bars are the standard deviations (SD), according to an embodiment of the disclosure.

FIG. 8 illustrates an instability of TTEY peptide during the digestion, according to an embodiment of the disclosure. Absolute peak areas of endogenous TTEY peptide as a function of the digestion time. At each time-point, 3 independent digests were injected in duplicate (n=6). Error bars represent the standard deviations (SD). Panel (A) 100 nmol/L calibrator r14K, (B) Sample S11, (C) sample S05 and (D) sample S07, according to an embodiment of the disclosure.

FIG. 9 illustrates digestion independent of concentration and isoform size for all candidate quantification peptides, according to an embodiment of the disclosure. Ratios of the peak areas of endogenous to Stable Isotope Labeled (SIL) peptide normalized to the digestion time T=18 h as a function of the digestion time. Panels (A) TPAY, (B) TPEN, (C) GISS, (D) GSFS, (E) LFLE and (F) TTEY Note: Not all ratios at 12 and 18 h could be calculated for TTEY because some peak areas were undetectable). At each time-point, 3 independent digests were injected in duplicate (n=6). Error bars are the standard deviations (SD), according to an embodiment of the disclosure.

FIG. 10 illustrates parallelism assessment of the 14K recombinant apo(a) calibrator for the candidate quantification peptides that did not meet the performance requirements of the final method, according to an embodiment of the disclosure. Each graph represents the ratios of peak areas of the endogenous to Stable Isotope Labeled (SIL) peptide as a function of the concentration in nmol/L. (A) GSFS and (B) LFLE peptides. The r14K calibration: diamond, the standard addition of known amounts of r14K to a given plasma sample: circle; the serial dilution of the same sample into “blank” plasma: square, according to an embodiment of the disclosure.

FIG. 11 illustrates an evaluation of the method inter-laboratory differences, according to an embodiment of the disclosure. Bland-Altman relative difference plot of the concentrations measured by micro-flow LC-MS/MS versus Lp(a) concentration measured by nano-flow LC-MS/MS on a set of 5 samples used in inter-laboratory comparison. Sample S07 (590 nmol/L) was also analyzed after a 2-fold dilution because of concentration outside the calibration range. Full line represents the average relative difference between the two methods, the dashed lines represent the ±1.96513 confidence interval, according to an embodiment of the disclosure.

FIG. 12 illustrates calibration curves for the three selected quantification peptides used in the comparison of LC-MSMS method to ELISA, according to an embodiment of the disclosure. Two independent LC-MS/MS quantification experiments to evaluate comparability of LC-MS/MS and ELISA gold standard were performed. Each calibration was prepared using fresh, independent aliquots of r14K. Each calibrator was digested and injected randomly in duplicate (n=4). Error bars represent the standard deviations (SD). Panel (A) TPAY, (B) TPEN, (C) GISS and (D) equations and R2 of the 1/× weighed calibration curves, according to an embodiment of the disclosure.

### DETAILED DESCRIPTION

With the renewed interest in Lp(a) as a causal risk factor for cardiovascular disease (CVD), poor method accuracy and lack of method standardization are the major obstacles for the clinical implementation of Lp(a) measurements. The complexity of Lp(a) makes it challenging to produce a primary reference material to anchor Lp(a) concentrations to SI units. Because apo(a) is the unique protein component of Lp(a) and its quantification is at the basis of most immunochemical measurements of Lp(a) in plasma, the inventors used this approach to develop an LC-MS/MS-based quantitative method that would meet the requirements of a reference method to be proposed as “candidate reference method” for the standardization of Lp(a) assays.

Requirements of a higher order reference method are defined by the JCTLM and the one requirement in particular is direct traceability to the SI through an unbroken chain of traceability, established through a suitable calibration strategy. The method should additionally provide equivalence of results between the calibration and the endogenous measurements (i.e., parallelism) and display high levels of accuracy and precision. Double isotope dilution LC-MS/MS is a method of choice for the absolute quantification of proteins using proteolytic peptides as surrogate measurands.

The methods of the disclosure address these requirements. Specifically, the inventors developed an accurate method for the quantification of Lp(a) in biological samples using a targeted LC-MS/MS approach with an isotope dilution calibration strategy.

Thus, one aspect of the disclosure provides a method for quantifying apolipoprotein(a) in a biological sample. Such method includes measuring an ion signal of at least two candidate peptides and an ion signal of their stable isotope labeled peptides in the biological sample by mass spectrometry analysis, normalizing the ion signal of at least two candidate peptides with the ion signal of stable isotope labeled peptides to obtain a measured average ion signal of the at least two peptides, and quantifying apolipoprotein(a) using the average ion signal of the at least two peptides and a reference amount of a calibrator.

The disclosure also provides kits useful for carrying out the methods of the disclosure as described herein. For example, such kit includes (i) two or more stable isotope labeled candidate peptides, and optionally (ii) one or more calibrators (such as the recombinant apo(a) or any other suitable calibrator material with accurately assigned apo(a) value).

As provided above, one aspect of the disclosure provides a method for determining if a subject is at risk for developing cardiovascular disease. As used herein, the term “cardiovascular disease” or “CVD,” generally refers to heart and blood vessel diseases, including atherosclerosis, coronary heart disease, cerebrovascular disease, and peripheral vascular disease. For example, the cardiovascular disease may be, but is not limited to, myocardial infarction, atherosclerosis, coronary artery disease, peripheral artery disease, heart failure, stroke, arterial thrombosis, calcific aortic valve disease, aortic stenosis, or venous thromboembolism.

To determine if a subject is at risk to develop cardiovascular disease, the method includes measuring an ion signal of at least two candidate peptides and an ion signal of their respective stable isotope labeled peptides in a biological sample; normalizing the ion signal of at least two candidate peptides with the ion signal of stable isotope labeled peptides to obtain an average ion signal of the at least two peptides; quantifying apolipoprotein(a) using the average ion signal of the at least two peptides and a reference amount of a calibrator; and comparing the amount of apolipoprotein(a) to a predetermined amount of lipoprotein(a), wherein a difference in the amount of apolipoprotein(a) in the biological sample and the predetermined amount of lipoprotein(a) is indicative of the risk to develop cardiovascular disease in the subject.

As would be understood by one of skill in the art, the predetermined amount of lipoprotein(a) refers to an amount of lipoprotein(a) indicative of CVD risk that may be generally established by American College of Cardiology and the American Heart Association (ACC/AHA), or a similar scientific body.

The amount of apolipoprotein(a) measured based on abundance of at least two peptides in the biological sample compared to the predetermined amount is indicative of the risk of developing cardiovascular disease in the subject. For example, in certain embodiments, an increased amount of apolipoprotein(a) in the biological sample compared to the predetermined amount is indicative of the risk of developing cardiovascular disease in the subject.

The biological sample of the disclosure as described herein may be a serum sample or a plasma sample. In certain embodiments, the biological sample is a plasma sample.

In certain embodiments, the candidate peptides of the disclosure are selected from TPEN, TPAY, and GISS. In the methods of the disclosure as described herein, the ion signal of at least two peptides selected from TPEN, TPAY, and GISS is measured. In certain embodiments of the methods of the disclosure as described herein, the ion signal of at least three peptides selected from TPEN, TPAY, and GISS is measured.

The method of the disclosure allows for measuring the ion signal of other peptides, such as LFLEPTQADIALLK (SEQ ID NO: 4) (LFLE) and GSFSTTVTGR (SEQ ID NO: 5) (GSFS). Therefore, in certain embodiments, the ion signal of LFLE and/or GSFS is measured.

Prior to measuring the ion signal of at least two peptides, in certain embodiments, the method of the disclosure as described herein further comprises first treating the biological sample with a protease that digests the apolipoprotein(a) into at least two peptides selected from TPEN, TPAY, and GISS. In certain embodiments, the protease is trypsin.

To determine the average ion signal of at least two candidate peptides, the methods of the disclosure further comprise adding the amount of two or more internal standards to the biological sample prior to measuring.

The internal standards may comprise a stable isotope labeled peptide to be measured. For example, if TPEN is to be measured, then the internal standard includes a stable isotope labeled TPEN. In another example, if TPEN, TPAY, and GISS are to be measured, then the internal standards include a stable isotope labeled TPEN, TPAY, and GISS. The two or more stable isotope labeled peptides, in certain embodiments, are ¹³C and/or ¹⁵N-labeled peptides. In one embodiment, the two or more peptides are labeled with ¹³C. In another embodiment, the two or more peptides are labeled with ¹⁵N. In another embodiment, the two or more peptides are labeled with both ¹³C and ¹⁵N.

In certain embodiments, the one of more calibrators comprises a calibrated reference sample selected from calibrated plasma sample (such as single or pooled), serum sample (such as single or pooled), and plasma or serum lacking Lp(a) spiked (i.e., modified) with a known amount of purified Lp(a) or 14K recombinant apo(a).

The calibrator of the disclosure may be obtained by assigning value to the reference sample (such as plasma or serum) based on a primary reference material. For example, such calibration may include: measuring an ion signal of at least two candidate peptides (such as two of TPEN, TPAY, and GISS) in the reference sample; and assigning the reference ion signal of the peptides based on the amount of the primary reference material and one of more internal standards comprising stable isotope labeled peptides to be measured. In certain embodiments, the primary reference material comprises a high-purity human recombinant apolipoprotein(a). For example, in certain embodiments, the high-purity human recombinant apolipoprotein(a) is a human recombinant apolipoprotein(a) containing 14 kringles IV (r14K) (SEQ ID NO: 7).

In certain embodiments, the methods of the disclosure as described herein further comprise comparing the ion signal of the at least two peptides to an amount of the two of more internal standards and to a reference amount of a calibrator. For example, the ion signal of the peptide(s) in the biological sample relative to the ion signal of two of more internal standards and to a reference amount of a calibrator may be indicative of an amount of apolipoprotein(a) in the biological sample.

The methods of the disclosure are carried out by mass spectrometry analysis using a mass spectrometer. The mass spectrometer refers to a device able to volatilize/ionize analytes to form gas-phase ions and determine their absolute or relative molecular masses. Suitable forms of volatilization/ionization are electrospray, laser/light, thermal, electrical, atomized/sprayed and the like, or combinations thereof. Suitable forms of mass spectrometry include, but are not limited to, ion trap instruments, quadrupole instruments, electrostatic and magnetic sector instruments, time of flight instruments, time of flight tandem mass spectrometer (TOF MS/MS), Fourier-transform mass spectrometers, and hybrid instruments composed of various combinations of these types of mass analyzers. These instruments may, in turn, be interfaced with a variety of sources that fractionate the samples (for example, liquid chromatography) and that ionize the samples for introduction into the mass spectrometer, including electrospray or nanoelectrospray ionization (ESI) or combinations thereof

In certain embodiments, the mass spectrometry analysis is a liquid chromatography-mass spectrometry (LC-MS). In certain embodiments, the mass spectrometry analysis is electrospray ionization mass spectrometry (ESI-MS), optionally comprising quadrupole time-of-flight (TOF), quadrupole-orbitrap mass spectrometry, triple quadrupole mass spectrometry, or tandem time of flight mass spectrometry.

Another aspect of the disclosure provides methods of determining a reference amount of apo(a) based on measurement of the average ion signal of at least two peptides in a reference sample. Such methods include:
calibrating the reference sample with a primary reference material comprising the high-purity human recombinant apolipoprotein(a);
identifying two or more peptides for quantification based on a group of factors to obtain the two or more peptides; and
determining the reference average ion signal of the two or more peptides normalized to ion signal of their respective stable isotope labeled peptides based on the amount of the primary reference material.

In certain embodiments, the group of factors to obtain the two or more peptides include, but is not limited to, a level of presence of the high-purity recombinant apolipoprotein(a) in a KIV-2 domain (for example, wherein the level of presence is one); a level of absence of an amino acid susceptible to a modification; a level of absence of homologous peptides in a human proteome; a level of absence of known human genetic mutations; and a combination thereof

In certain embodiments, the high-purity human recombinant apolipoprotein(a) is r14K (SEQ ID NO: 7). The reference sample, for example, may be selected from plasma sample (such as single or pooled), serum sample (such as single or pooled), and plasma or serum lacking Lp(a) spiked (i.e., modified) with a known amount of purified Lp(a) or 14K recombinant apo(a). In certain embodiments, the two or more peptides identified for quantifications are any two of candidate peptides as described herein (such as the one or more peptides selected from TPEN, TPAY, and GISS).

### EXAMPLE

The methods of the disclosure are illustrated further by the following examples, which are not to be construed as limiting the disclosure in scope or spirit to the specific methods described in them.

### Materials and Methods

### Human Samples

Blood samples from individual donors were collected in lavender top 10 mL K2EDTA tubes. After blood collection, the tubes were inverted several times and let sit on crashed ice for 20-30 minutes. The blood was then centrifuged at 1,500 g for 10 minutes at 4 ° C. Isolated plasma was aliquoted in 2 mL conical polypropylene cryovials and fresh-frozen at −80 ° C. A plasma with a low concentration of Lp(a) (4.9 nmol/L by ELISA), further referred to as “blank” plasma, was used as blank matrix. The use of human specimens was approved by the Human Subjects Division at the University of Washington. All donors provided a written informed consent.

### Primary Reference Material

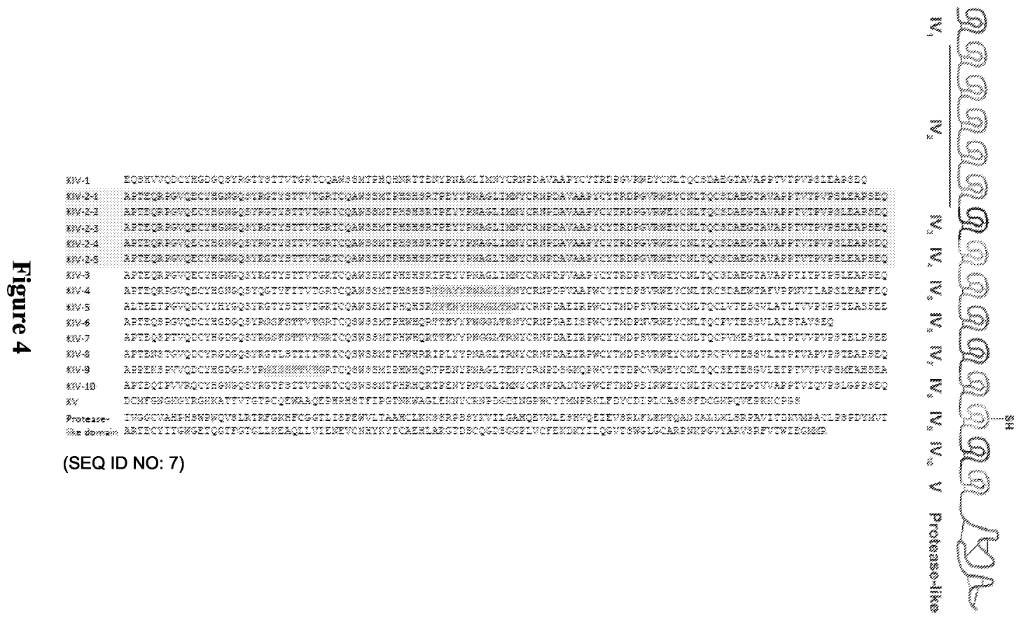

A high-purity human recombinant apo(a) with 14 kringles IV (r14K) was selected as primary reference material to calibrate the assay. The full sequence of r14K is provided in FIG. 4. The r14K apo(a) was expressed in human embryonic kidney 293 (HEK293) cells, stably transfected with a 14K-pRK5 expression vector, purified by Lys-Sepharose affinity chromatography, aliquoted and stored at −80° C. This expression protocol ensured the proper folding and glycosylation of the recombinant apo(a), which retained the same structural and functional characteristics as the endogenous protein. The size and purity were ascertained by agarose gel electrophoresis, SDS-PAGE electrophoresis, electrospray differential ion mobility analysis and anion-exchange fast protein liquid chromatography.

### Determination of the Concentration of the Primary Reference Material

Concentration of the r14K was determined by amino-acid analysis at the National Institute of Standards and Technology (NIST) using a method calibrated with pure higher-order amino-acid (AA) reference standards from the National Metrology Institute of Japan and registered in the Joint Committee for Traceability in Laboratory Medicine (JCTLM) database. Gas-phase hydrolysis of the protein was performed using two different hydrolysis conditions and six AA were quantified by LC-MS/MS. Triplicate independent measurements were performed on two different aliquots of the purified r14K. Uncertainties of the 6 AA measurement obtained with the two protocols were overlapping and therefore the reported value for r14K concentration was calculated across all six AAs and both sets of results.

### Method Calibration

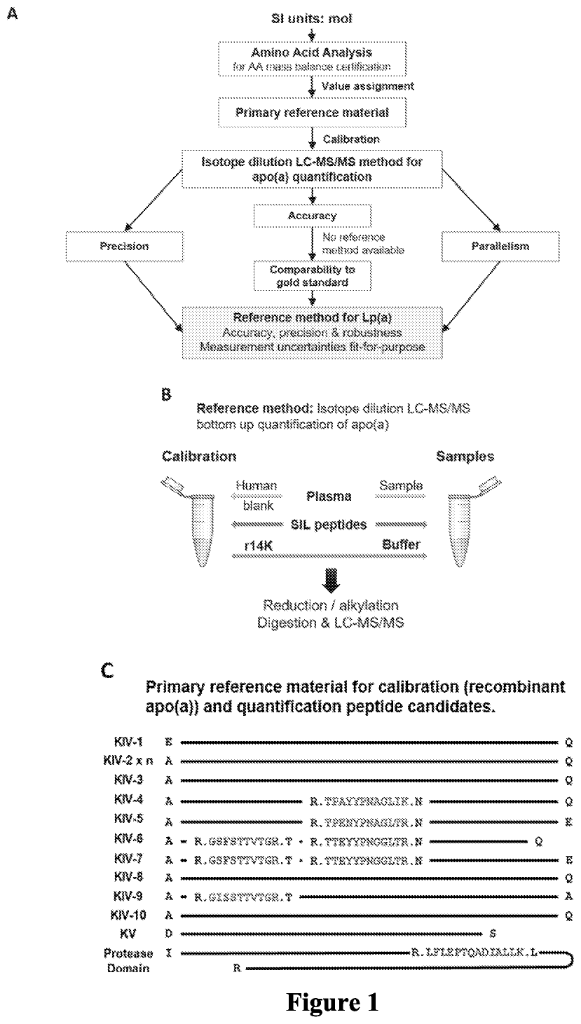

Calibrators were prepared using the r14K spiked into “blank” plasma. Double isotope dilution was used for quantification by including the same amount of pure synthetic stable isotope labeled (SIL) peptides in calibrators and in plasma samples. Briefly, r14K was diluted gravimetrically to 6 concentration levels for final calibrator concentrations of 20 to 400 nmol/L and further supplemented with SIL peptides at a final concentration of 100 nmol/L (FIG. 1B). All calibrator stocks and intermediates were prepared fresh for each assay. The calibration curve was constructed using linear regression with 1/× weighting without including the blank or the origin.

### Digestion Protocol

Samples were prepared by combining 10 μL of plasma, 40 μL of 100 mmol/L ammonium bicarbonate and 20 μL of the SIL peptide working solution (FIG. 1B). After denaturation in 0.5% (w/v) sodium deoxycholate, samples were reduced with dithiothreitol, alkylated with iodoacetamide and digested with trypsin for 18 h at 37° C. Digested samples were acidified with formic acid to precipitate sodium deoxycholate and supernatants were analyzed fresh in LC-MS/MS.

### Selection of Peptides for Quantification

Candidate peptides for quantification were identified using data independent analysis on trypsin digests of both pure r14K and Lp(a) enriched samples, i.e. reconstituted pellet after apolipoproteinB-100 (apoB-100) precipitation from plasma with 300 nmol/L Lp(a) concentration. From 18 peptides reliably measured in data independent analysis, 6 candidate quantification peptides were selected based on the following criteria: 1) not present in KIV-2 domain of apo(a), 2) absence of AA susceptible to ex-vivo modifications (methionine, cysteine, or terminal glutamic acid or glutamine), 3) absence of homologous peptides in the human proteome, and 4) absence of known human genetic mutations (FIG. 1C).

### LC-MS/MS Method

The LC-MS/MS analysis was performed on a Waters Nano-Acquity UPLC system (Waters) coupled to a Thermo ALTIS triple quadrupole mass spectrometer with electrospray ionization (Thermo Fisher). For each peptide and their SIL analogs, measured transitions were summed and a ratio of the chromatographic peak area of endogenous to SIL peptide was calculated in Skyline. While Clinical and Laboratory Standards Institute (CLSI) C62-A recommends that clinical LC-MS/MS methods use a single transition for quantification with a second transition as qualifier, we averaged at least 3 transitions for each peptide to increase the robustness of the method. All initial data processing was performed using Skyline, quantification was performed in Excel.

### Method Development and Validation

Comparability of the digestion kinetics between r14K and endogenous apo(a) was verified over an 18 h time-course experiment with 3 individual plasma samples with Lp(a) concentrations of 130, 150, and 590 nmol/L and major apo(a) isoform sizes 30, 21, and 12 MV respectively, and with a 100 nmol/L r14K in “blank” plasma (Table 1). For each time-point, samples were digested in triplicate and analyzed in duplicate by LC-MS/MS (n=6).

Table 1 shows clinical specimens used for the evaluation of digestion time-course. Samples were selected based on Lp(a) concentration measured by ELISA and isoform profiles determined by agarose gel electrophoresis. Relative proportions are indicated for each sample. A 100 nmol/L solution of the human recombinant protein calibrator r14K was included to ensure that the recombinant protein followed the same digestion kinetics as endogenous apo(a).

TABLE 1

| | C_{ELISA} | Isoform 1 | | Isoform 2 | |
|---|---|---|---|---|---|
| Samples | (nmol/L) | Size | Proportion | Size | Proportion |
| r14K | 100 | 14 | 100% | — | — |
| S11 | 130 | 29 | 65% | 32 | 35% |
| S05 | 180 | 19 | 75% | 22 | 25% |
| S07 | 590 | 12 | 100% | — | — |

Limits of detection (LODs) were calculated as the “blank” plasma response plus 2 standard deviations (SD). The lower limits of quantification (LLOQs) were estimated in several experiments with a maximum allowable bias of 15% and a maximum allowable coefficient of variation (CV) of 20% at LLOQ (CLSI C62-A). Repeatability and intermediate precision were assessed on five samples with concentrations (by ELISA) ranging from 20.3 nmol/L to 590.5 nmol/L (Table 2), assayed in triplicate on 3 different days, one week apart. Intra-day and inter-day CVs were calculated.

Table 2 lists the clinical specimens used for the evaluation of the intermediate precision of the LC MS/MS method. Lp(a) concentrations were measured by the designated comparison ELISA. Isoforms were determined by agarose gel electrophoresis. Relative proportions (rounded at 5%) of each isoform are indicated for each sample.

TABLE 2

| | C_{ELISA} | Isoform 1 | | Isoform 2 | |
|---|---|---|---|---|---|
| Samples | (nmol/L) | Size | Rel. % | Size | Rel. % |
| S01 | 20.3 | 16 | 95% | 28 | 5% |
| S02 | 38.1 | 24 | 90% | 17 | 10% |
| S03 | 81.7 | 23 | 55% | 21 | 45% |
| S05 | 179.9 | 19 | 75% | 22 | 25% |
| S07 | 590.5 | 12 | 100% | — | — |

### Parallelism

Parallelism of responses between r14K and endogenous apo(a) was assessed using a plasma with 150 nmol/L Lp(a) determined by ELISA and a 3-step approach: 1) standard additions of r14K to the plasma sample, 2) serial dilutions of the same plasma sample with “blank” plasma and 3) preparation of a calibration curve using r14K in “blank” plasma. Parallelism of the 3 regression lines was assessed in 2 independent assays with freshly prepared samples and calibrators by comparing the slopes and their respective 95% confidence intervals (CI).

### Assessment of Comparability to the Gold Standard ELISA

In the absence of a reference method, and per JCTLM recommendations, comparability of LC-MS/MS results against the gold standard ELISA was evaluated on a set of 64 individual well-characterized samples. The ELISA was performed as previously reported in duplicate, on 6 different days, and the SD (n=12) was calculated. For the LC-MS/MS, 2 independent assays were performed as described earlier and samples were digested and injected once per each assay. Methods were compared using weighted-Deming and Pearson least squares regression models. Relative differences to ELISA were evaluated using Bland-Altman difference plot.

### Reagents and Materials

Ammonium bicarbonate (AmBic), formic acid (FA) and sodium deoxycholate (SDC) were purchased from Sigma Aldrich. Dithiothreitol (DTT) and iodoacetamide (IAA) were purchased from BioRad. Protein reduction and alkylation was performed in 2 mL 96-well deep-well microplates from Greiner Bio-One. Digestion was done in 96-well V-bottom 500 μL polypropylene deep-well plates from Axygen using proteomics sequencing grade Trypsin (Promega™ Gold). All solvents used were LC-MS/MS grade.

As a matrix for calibrators and diluted samples in the parallelism study, a plasma with extremely low concentration of Lp(a) (<5 nmol/L ELISA) was used and is referred to as “blank” plasma in the following paragraphs. All clinical samples used for quality control, validation, and method comparison to ELISA were obtained from individual donors by venipuncture and were drawn into lavender top EDTA tubes containing 1.8 mg EDTA/mL blood (Becton Dickinson). Blood was gently mixed by inversion and left on ice for 20 min prior to centrifugation. Fresh plasma was collected, 0.5 mL were aliquoted in 2 mL cryovials, frozen and stored at −80° C. until use.

### Preparation of the Primary Reference Material

A high purity human recombinant apo(a) was selected as a primary reference material to calibrate the assay. The expression of the human recombinant apo(a) was performed as previously reported in detail (Willeit P et al., Lancet 2018; 392:1311-20). Briefly, recombinant apo(a) was expressed in human embryonic kidney cells 293 (HEK293) stably transfected with a 14K-pRKS expression vector expressing 14 apo(a) kringle IV domains (r14K) from a cytomegalovirus promoter. This expression protocol ensures the proper folding and glycosylation of the recombinant apo(a) which retains the same structural and functional characteristics as the endogenous protein. For purification, filtered culture medium was loaded onto a 50 mL Lysine-Sepharose column equilibrated in a phosphate buffer saline pH 7.4 containing 500 mmol/L NaCl. After purification, r14K was concentrated 10-fold using a centrifugal concentrator with a molecular weight cut-off of 10 kDa, dialyzed against three changes of 4 L HEPES buffered saline buffer and stored in aliquots at −80° C. in 2 ml cryovials.

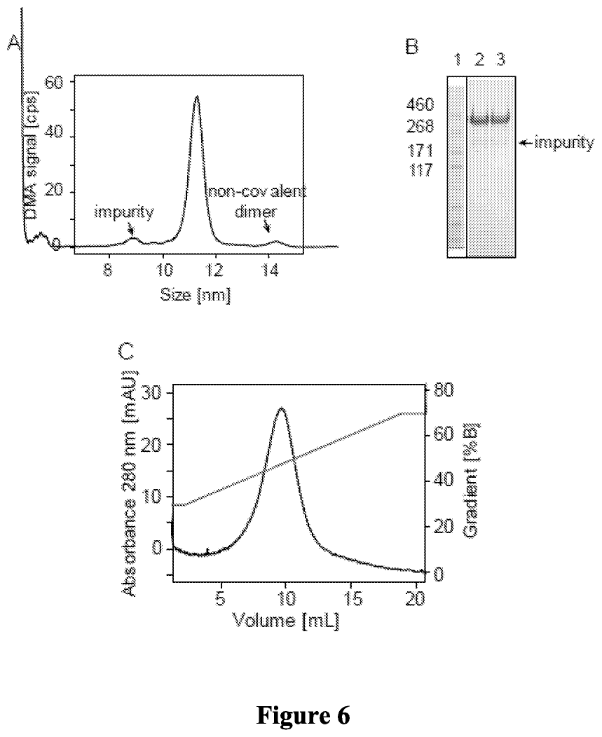

The size of r14K was verified by a high-sensitivity agarose gel electrophoresis performed as previously described in detail (Tsimikas S. J Am Coll Cardiol 2017; 69:692-711). Purity was assessed as first intention by several orthogonal methods: gel electrophoresis on a 3-8% Tris-Acetate gel, stained with Coomassie Blue and HiMark™ pre-stained protein standards (ThermoFisher, #LC5699—purity calculated from densitometric analysis), by electrospray-differential ion mobility analysis (purity calculated from the peak area under the curve of monomer and dimer peaks), and lastly by anion-exchange fast-protein liquid chromatography on MiniQ 4.6/50 column in 20 mmol/L HEPES pH 8 (buffer A) and 1 mol/L NaCl in 20 mmol/L HEPES pH 8 (buffer B) with a linear gradient 30-70% B in 20 column volumes with flow-rate 1 mL/min. (FIG. 6).

### Determination of the Concentration of the Primary Material

Concentration of the r14K was determined by amino-acid analysis at the National Institute of Standards and Technology using a method calibrated with pure higher-order amino-acid (AA) reference standards from the National Metrology Institute of Japan and registered in the Joint Committee for Traceability in Laboratory Medicine database.

After dilution in ultrapure water, gas-phase hydrolysis of the protein was performed using 6N HC1 in two different hydrolysis conditions: 130° C. for 48 h, and 140° C. for 71 h. Hydrolyzed samples were analyzed by targeted LC-MS/MS in multiple reaction monitoring mode, and six AA were quantified: phenylalanine (Phe), leucine (Leu), isoleucine (Ile), valine (Val), alanine (Ala) and proline (Pro). Two fragmentation transitions were used for each AA except for Ala and Pro for which only one transition was used for quantification.

AAs were quantified using a double isotope dilution measurement system involving stable isotope labelled (SIL) AAs (¹³C and/or ¹⁵N) sourced from CIL. Briefly, this strategy involves spiking samples with a mixture of the SIL-AAs before hydrolysis while also preparing a series of external calibrators made of pure AAs from National Metrology Institute of Japan spiked with the identical SIL-AA mixture to quantify the samples. Triplicate independent measurements were performed on two different aliquots of the purified r14K using two different hydrolysis methods. Uncertainties of the measurement of the 6 AAs obtained using the two hydrolysis methods were overlapping and therefore the reported value for r14K concentration was calculated across all six AAs analytes and both sets of hydrolysis.

### Preparation of Calibrator and Stable Isotope Labelled Peptide Standards

To minimize variability associated with the LC-MS/MS and to improve method precision and accuracy, SIL peptide analogs were used as internal standards. Highly pure synthetic SIL peptides labeled at C-terminal Arg or Lys residues with ¹³C and ¹⁵N (¹³C₆/¹⁵N₂-Lys, ¹³C₆/¹⁵N₄-Arg) (NEPTune grade, >98% purity, >99% isotopic enrichment) were purchased solid from New England Peptide, Inc. Each peptide was individually reconstituted in 50% acetonitrile (ACN)/0.1% FA to prepare 100 μmol/L stock solutions that were aliquoted and stored at −80° C. For each assay, an aliquot of each individual SIL peptide stock solution was diluted to 2 μmon in 5% ACN/0.1% FA. SIL peptides were combined and further diluted in 100 mmol/L AmBic to prepare a working solution with a final concentration of 50 nmol/L of each peptide.

Quantification was performed using a double ID strategy: 1) a set of calibrators was prepared fresh with different ratios of r14K to SIL peptide and, 2) plasma samples were spiked with the same SIL peptide working solution to the same final concentration. To prepare the calibration curve, an aliquot of the r14K stock solution was thawed and diluted to 0.5 μmon in 100 mmol/L AmBic. This intermediate stock was gravimetrically diluted in 100 mmol/L AmBic into six different intermediate calibrators with concentrations ranging from 100 to 2.5 nmol/L. 40 μL of each intermediate calibrator was then spiked with 20 μL of the SIL peptide working mix. 10 μL of “blank” plasma (<5 nmol/L of apo(a)) was added to each calibrator. Calibrators were then processed like samples. All calibrator stocks and intermediates were prepared fresh for each assay.

### Digestion Protocol

Samples were prepared by combining 10 μL of plasma, 40 μL of 100 mmol/L AmBic and 20 μL of the SIL peptide working solution. Samples and calibrators (prepared as described above) were mixed in 2 mL 96-well deep-well plates with 75 μL of 1% SDC (w/v) , reduced with 5 μL of 250 mmol/L DTT at 90° C. for 60 minutes at 500 rpm in a Thermomixer™ and alkylated with 10 μL of 500 mmol/L iodoacetamide (IAA) for 30 minutes in the dark, at room temperature. Alkylation was quenched with additional 5 μL of 250 mmol/L dithiothreitol followed by dilution to 1 mL with 0.5% SDC (w/v) in 100 mmol/L AmBic. Fifty μL were transferred to a 96-well V-bottom 500 μL microtiter plate and diluted in equal volume of 0.5% SDC (w/v). Two μg of sequencing grade Promega trypsin dissolved in 1 mmol/L HCl was added and samples were digested overnight (18 h) at 37° C. on a Thermomixer (700 rpm). Digestion was quenched with 20 μL of 20% (v/v) FA and after 15 min incubation at room temperature with gentle mixing (300 rpm), the plate was centrifuged at 4000 g for 30 min at 4° C. Forty μL of the supernatant were transferred to a PCR-microtiter plate, diluted in 80 μL of 1% (v/v) ACN/0.1% (v/v) FA and analyzed fresh by LC-MS/MS.

### Data Independent Analysis for Peptide Selection

Candidate proteotypic peptides for quantification were identified using data independent analysis on trypsin digests of both Lp(a) enriched samples (reconstituted pellet after apoB-100 precipitation from plasma) and purified r14K. DIA analysis was performed on a tribrid Thermo Fusion Lumos mass spectrometer with electrospray ionization (Thermo Fisher Scientific). Tryptic digests were desalted on a C18 trapping column (Reprosil-Pur 120 C18-AQ, 5 μm, 0.1×40 mm, Dr. Maisch HPLC) (trapping flow rate 4 μL/min), separated on a capillary analytical column (Reprosil-Pur 120 C18-AQ, 5 μm, 250×0.075 mm, Dr. Maisch HPLC) with a multi-step linear gradient 1-5%B in 2 min, 5-25% in 50 min, 25-35% in 10 min followed by a ramp to 80%B and re-equilibration (A—0.1% (v/v) FA in water, B—acetonitrile, 0.1% (v/v) FA, flow rate of 0.4 μL/min) using a nano-Acquity UPLC (Waters Corporation).

DIA was performed as follows: 1) MS1 scan (395-100 Da) at resolution 120,000, maximum injection time 50 ms; 2) 60 MS/MS scans on 10 Da mass windows across 400-100 Da range (resolution 15,000 with loop time 3 s and maximum injection time 22 ms, high-energy C-trap dissociation at normalized collision energy 30%). Data were further processed using Skyline.

### LC-MS/MS Method

The LC-MS/MS analysis was performed on an LC-MS/MS Waters Nano-Acquity UPLC system (Waters Corporation) coupled to a Thermo ALTIS triple quadrupole mass spectrometer with electrospray ionization (Thermo Fisher Scientific). Two μL of sample were desalted on a C18 trapping column (Reprosil-Pur 120 C18-AQ, 5 μm, 0.1×40 mm, Dr. Maisch HPLC) and eluted onto an analytical C18 capillary column (Reprosil-Pur 120 C18-AQ, 5 μm, 250×0.075 mm, thermostated at 35° C.). Chromatographic method consisted of 5 min trapping at a flow rate of 4 μL/min followed by a multi-step linear gradient of 1-5% B (2 min), 5-25% B (20 min), 25-35% B (2 min) followed by a column wash at 80% B and re-equilibration (A: 0.1% FA (v/v) in water; B: acetonitrile, 0.1% FA (v/v); flow rate of 0.4 μL/min) with total run time of 47 min. Analyses were performed in selected reaction monitoring mode with 3 min scheduled retention time windows for each transition and a 10 ms dwell time. Collision energy (CE) was optimized for each peptide to maximize response using collision energy optimization facilitated by Skyline software and quantitative transitions were selected to avoid matrix interferences. For each peptide and their SIL analogs, measured transitions were summed and a ratio of the chromatographic peak area of endogenous to SIL peptide was calculated in Skyline to provide a peptide response. Calibration curve was constructed using linear regression with 1/× weighting not including blank or origin.

Because the nominal concentration of the “blank” plasma was below the limit of quantification, we did not correct peak areas of endogenous peptides with the signal measured in the “blank”. However, the effect of subtracting the “blank” signal from calibrators and samples was investigated. Differences with and without correction were negligible at all levels and did not have influence on the final Lp(a) concentrations (Table 3).

Table 3 is a comparison of the LC-MS/MS quantification of the 5 validation samples and SRM-2B using calibration curves constructed with and without correction for the “blank” plasma Lp(a) concentration (measured by ELISA).

TABLE 3

| | | Without Blank Correction | | | | | With Blank Correction | | | | |
|---|---|---|---|---|---|---|---|---|---|---|---|
| | | | | | 3pep | | | | | 3pep | |
| | ELISA | TPAY | TPEN | GISS | mean | CV | TPAY | TPEN | GISS | mean | CV |
| | (nmol/L) | (nmol/L) | (nmol/L) | (nmol/L) | (nmol/L) | (%) | (nmol/L) | (nmol/L) | (nmol/L) | (nmol/L) | (%) |
| S01 | 20.3 | 24.3 | 21.2 | 20.1 | 21.9 | 9.7% | 23.7 | 21.1 | 19.9 | 21.6 | 9.1% |
| S02 | 38.1 | 40.3 | 41.2 | 37.9 | 39.8 | 6.1% | 405 | 41.0 | 38.1 | 39.8 | 5.8% |
| S03 | 81.7 | 85.0 | 76.5 | 77.9 | 79.8 | 5.3% | 85.0 | 76.8 | 78.0 | 79.9 | 5.1% |
| S05 | 179.9 | 199.2 | 173.0 | 175.8 | 182.7 | 7.3% | 198.9 | 173.4 | 176.0 | 182.8 | 7.2% |
| S07 | 590.5 | 652.9 | 611.9 | 620.6 | 628.5 | 3.0% | 652.3 | 611.6 | 619.7 | 627.9 | 3.0% |
| PR | 107.0 | 108.7 | 102.0 | 101.7 | 104.1 | 4.5% | 108.3 | 102.3 | 101.6 | 104.0 | 4.1% |

Relative difference between corrected and
non-corrected LC-MS/MS values (%)

| | TPAY | TPEN | GISS | 3pep mean |
|---|---|---|---|---|
| | (nmol/L) | (nmol/L) | (nmol/L) | (nmol/L) |
| S01 | −2.5% | −0.5% | −0.9% | −1.4% |
| S02 | 0.4% | −0.5% | 0.5% | 0.1% |
| S03 | 0.0% | 0.4% | 0.1% | 0.1% |
| S05 | −0.1% | 0.2% | 0.1% | 0.0% |
| S07 | −0.1% | −0.1% | −0.1% | −0.1% |
| PRM | −0.4% | 0.3% | −0.1% | −0.1% |

While Clinical and Laboratory Standards Institute (CLSI) C62-A recommends for LC-MS/MS methods used in the clinical laboratory to use a single transition for quantification with a second transition as qualifier, we averaged at least 3 transitions for each peptide to increase the robustness of the method. All initial data processing was performed using Skyline with quantification performed in Excel.

### Digestion

Consistency of the digestion kinetics was verified with three individual plasma samples from donors with different apo(a) concentrations and isoform sizes, and 100 nmol/L r14K prepared in “blank” plasma. Digestion was performed for: 30 min, 1 h, 2 h, 4 h, 8 h and 18 h. For each time-point, samples were digested in triplicate and injected in duplicate in LC-MS/MS (n=6). All samples were analyzed in a single experiment and injected on LC-MS/MS in a random-order injection sequence. To verify stability of the signal over time, a 100 nmol/L pure r14K digested for 18 h was injected in triplicate at five different times (after 35h, 68 h, 98 h and 125 h).

Digestion completeness was assessed for each candidate quantification peptide by plotting the ratios of endogenous to SIL peptide as a function of the digestion time. Stability of the peptides under digestion conditions was assessed by inspecting the absolute areas normalized to t=18h as a function of digestion time. Finally, to evaluate the influence of apo(a) isoform size on digestion completeness, ratios to SIL peptides were normalized to the average ratio at t=18h for each peptide.

### Linearity, Repeatability, and Intermediate Precision

Limits of detection (LOD) for the candidate quantification peptides were calculated as the “blank” plus 2 SDs. The limits of quantification (LOQ) were estimated in several experiments using Skyline with a maximum allowable bias at LLOQ of 15% and a maximum allowable CV of 20%, according to CLSI recommendations C62-A.

Repeatability and intermediate precision were assessed for all candidate quantification peptides on five different samples with concentrations ranging from 20.3 nmol/L to 590.5 nmol/L. Repeatability and intermediate precision for the 590.5 nmol/L sample that was outside the calibration range were assessed on both the undiluted sample and on a 1:1 dilution in “blank” plasma. Samples were digested in triplicate and each digest was injected in triplicate (n=9 per sample). Three independent assays were done on separate weeks, with freshly prepared calibrators, SIL peptides and samples (n=27 overall). The study was performed in conditions of intermediate precision, sample preparation and measurements being performed by the same operator on the same instrument in the same laboratory.

Data were first tested for outliers using one and two values Grubbs test with 95% confidence interval (CI). Statistical outliers were excluded, but isolated values were kept. Technical repeatability was calculated as the coefficient of variation (CV) on the three injections performed for each digested sample. Intra-day CVs and inter-day CVs were calculated.

### Reproducibility and Inter-Laboratory Validation

Inter-laboratory validation was performed at University of Washington Laboratory Medicine on a normal-flow LC-MS/MS using reagents described above with the following exceptions: ammonium bicarbonate was from Sigma-Aldrich (30970-100G), iodoacetamide from Acros Organics (AC12227-0050), dithiothreitol from Bio-Rad (1610611), Suprapure FA from EMD-Millipore (11.1670.1000), and hydrochloric acid (1 N) (SA48-4), LC-MS grade dimethylsulfoxide (DMSO) (85190), LC-MS grade methanol (A456), LC-MS grade acetonitrile (A955), and LC-MS grade water (W64) from Fisher Scientific.

Sample preparation was performed following the same protocol described above with the following exceptions: after sample digestion and acidification, plates were centrifuged at 3720 g for 30-60 min. If visible particulates remained in solution, samples were transferred to 1.5 mL Safe-Lock Eppendorf tubes (P/N 022431081) and centrifuged at 14,100 g for 15-30 min at 2-8° C. until supernatant was free of particulates. Forty μL were transferred to a Waters Sample Collection Plate (P/N 0006481604) and diluted with 80 μL of 1% (v/v) ACN/0.1% (v/v) FA. Assays 1 and 2 were analyzed fresh, plate 3 was stored at −20° C. for 2 weeks prior to analysis.

Samples were analyzed with a targeted LC-MS/MS method on a Waters Acquity UPLC and XEVO TQ-S with MassLynx 4.2 software. Calibrators, controls, and test samples were injected (25 μL) onto a Waters Acquity UPLC HSS T3 column (2.1×50 mm 1.8 μm, P/N 186003538) at 45° C., equipped with a Waters vanguard HSS T3 guard column (P/N 186003976). Peptides were eluted at a flow rate of 0.3 mL/min with a gradient from 2-98% mobile phase B over 7 min (mobile phase A: 2% DMSO, 0.1% FA in water and mobile phase B: 2% DMSO, 0.1% FA in MeOH). The column was washed briefly at 95% B and re-equilibrated at starting conditions for 1.8 min (total LC run time of 10 min). The capillary voltage was 1.0 KV, cone voltage was 35, desolvation temperature was 500° C., desolvation gas was 1000 L/hr, and cone gas was 150 L/hr. The collision cone voltage and transition dwell times are listed in Table 4. Peptides were scheduled with approximately 1 min windows.

Table 4 provides instrument parameters for the reproducibility study on micro-flow Waters Acquity UPLC system. Parameters were optimized using multiple injections with step functions. Transitions were chosen to avoid chromatographic interference.

TABLE 4

| | Precursor | Fragment | | Collision |
|---|---|---|---|---|
| Peptide | (m/z) | (m/z) | Dwell(s) | (eV) |
| LFLEPTQADIALLK | 786.94 | 1070.27 | 0.030 | 28 |
| (SEQ ID NO: 4) | | | | |
| LFLEPTQADIALLK (heavy) | 790.91 | 1078.21 | 0.030 | 28 |
| (SEQ ID NO: 8) | | | | |
| GISSTTVTGR | 490.04 | 808.86 | 0.013 | 17 |
| (SEQ ID NO: 3) | | 721.78 | 0.013 | 17 |
| GISSTTVTGR (heavy) | 495.00 | 818.79 | 0.013 | 17 |
| (SEQ ID NO: 9) | | 731.71 | 0.013 | 17 |
| TPAYYPNAGLIK | 654.76 | 876.04 | 0.030 | 23 |
| (SEQ ID NO: 2) | | 712.86 | 0.030 | 23 |
| TPAYYPNAGLIK (heavy) | 658.73 | 883.98 | 0.030 | 23 |
| (SEQ ID NO: 10) | | 720.80 | 0.030 | 23 |
| TPENYPNAGLTR | 667.22 | 728.82 | 0.013 | 24 |
| (SEQ ID NO: 1) | | | | |
| TPENYPNAGLTR (heavy) | 672.19 | 738.75 | 0.013 | 24 |
| (SEQ ID NO: 11) | | | | |

Data analysis was performed using Skyline and Excel. Similar to the intermediate precision study, data was tested for outliers using one and two values Grubbs test with 95% CI. Statistical outliers were excluded, but isolated values were kept. Technical repeatability of the method was calculated as the CV on the three injections performed for each digested sample. Intra-day CVs, inter-day CVs and inter-lab CVs were calculated to evaluate performance and reproducibility of the transferred method (Table 5).

Table 5 shows repeatability and reproducibility of the LC MS/MS method developed and validated on a nano-flow LC MS/MS system in Laboratory #1 and transferred to a normal-flow high-throughput LC MS/MS system in Laboratory #2. Intra-day precision is the variation coefficient (CV) of 3 independent digestions, injected in triplicate the same day (n=9). Inter-day precision is the CV over 3 days (n=27). Inter-laboratory precision is the CV across all measurements at both laboratories.

TABLE 5

| | | Univeristy of Washington - Lab #1 (nano-flow) | | | | | | | | | |
|---|---|---|---|---|---|---|---|---|---|---|---|
| | | Intra-day precision (n = 9) | | | | Inter-day precision (n = 27) | | | | University of Washington - Lab #2 (normal-flow) | |
| | | | | | 3 Pep. | | | | 3 Pep. | Intra-day precision (n = 9) | |
| | Assay | TPAY | TPEN | GISS | Av. | TPAY | TPEN | GISS | Av. | TPAY | TPEN |
| S01 | 1 | 5.2% | 3.9% | 5.2% | 6.8% | 7.3% | 4.8% | 8.9% | 7.9% | 12.3% | — |
| | 2 | 6.7% | 6.2% | 7.3% | 7.7% | | | | | 25.2% | 12.6% |
| | 3 | 4.8% | 4.0% | 7.3% | 6.3% | | | | | 23.2% | 15.3% |
| S02 | 1 | 4.6% | 5.5% | 3.5% | 4.9% | 7.1% | 6.0% | 5.2% | 7.1% | 7.6% | — |
| | 2 | 6.4% | 7.0% | 4.3% | 7.5% | | | | | 12.0% | 16.3% |
| | 3 | 7.4% | 5.7% | 4.7% | 7.3% | | | | | 22.6% | 17.8% |
| S03 | 1 | 2.6% | 2.7% | 2.8% | 5.6% | 3.8% | 4.1% | 5.0% | 5.9% | 6.4% | — |
| | 2 | 3.2% | 4.8% | 4.0% | 5.9% | | | | | 11.0% | 12.4% |
| | 3 | 3.8% | 5.0% | 3.4% | 5.5% | | | | | 16.8% | 15.1% |
| S05 | 1 | 2.3% | 3.6% | 2.2% | 6.3% | 3.0% | 3.6% | 2.5% | 7.1% | 5.2% | — |
| | 2 | 2.7% | 3.8% | 2.4% | 7.6% | | | | | 8.0% | 9.1% |
| | 3 | 2.7% | 3.6% | 2.9% | 7.6% | | | | | 12.5% | 6.0% |
| 507 | 1 | 2.8% | 2.0% | 2.5% | 4.8% | 3.7% | 1.8% | 3.5% | 5.2% | 5.7% | — |
| | 2 | 3.9% | 1.8% | 3.0% | 5.4% | | | | | 11.2% | 7.2% |
| | 3 | 3.4% | 1.8% | 2.9% | 5.0% | | | | | 14.3% | 10.3% |
| S07-Dil | 1 | 3.0% | 2.3% | 4.2% | 5.5% | 3.5% | 3.9% | 4.8% | 5.8% | 3.0% | — |
| | 3 | 2.4% | 4.1% | 4.0% | 5.4% | | | | | 10.7% | 8.1% |
| | 3 | 2.4% | 4.1% | 3.5% | 5.3% | | | | | 12.1% | 8.1% |

| | | | | | | |
|---|---|---|---|---|---|---|
| Average CV (%) | 4.0% | 4.0% | 3.9% | 6.2% | 12.2% | 11.5% |

| | | University of Washington - Lab #2 (normal-flow) | | | | | | | | | |
|---|---|---|---|---|---|---|---|---|---|---|---|
| | | Intra-day precision (n = 9) | | Inter-day precision (n = 27) | | | | Inter-laboratory precision (n = 54) | | | |
| | | | 3 Pep. | | | | 3 Pep. | | | | 3 Pep. |
| | Assay | GISS | Av. | TPAY | TPEN | GISS | Av. | TPAY | TPEN | GISS | Av. |
| S01 | 1 | — | — | 20.4% | 14.1% | 19.4% | 18.5% | 15.2% | 9.7% | 14.4% | 9.2% |
| | 2 | 21.1% | 20.9% | | | | | | | | |
| | 3 | 18.8% | 18.6% | | | | | | | | |
| S02 | 1 | — | — | 17.2% | 17.2% | 9.8% | 15.3% | 13.1% | 11.6% | 7.5% | 8.6% |
| | 2 | 11.4% | 13.1% | | | | | | | | |
| | 3 | 8.7% | 17.3% | | | | | | | | |
| S03 | 1 | — | — | 11.8% | 13.4% | 14.2% | 14.6% | 8.7% | 9.0% | 10.7% | 7.5% |
| | 2 | 15.7% | 14.2% | | | | | | | | |
| | 3 | 13.6% | 16.8% | | | | | | | | |
| S05 | 1 | — | — | 9.1% | 7.5% | 11.3% | 11.1% | 6.8% | 6.0% | 7.3% | 4.6% |
| | 2 | 9.4% | 10.4% | | | | | | | | |
| | 3 | 12.2% | 12.6% | | | | | | | | |
| 507 | 1 | — | — | 11.5% | 8.7% | 12.0% | 11.5% | 8.5% | 6.1% | 8.3% | 6.1% |
| | 2 | 16.2% | 12.2% | | | | | | | | |
| | 3 | 6.3% | 11.5% | | | | | | | | |
| S07-Dil | 1 | — | — | 9.9% | 8.5% | 10.0% | 10.5% | 7.7% | 7.2% | 7.4% | 5.7% |
| | 3 | 9.7% | 11.4% | | | | | | | | |
| | 3 | 9.9% | 10.7% | | | | | | | | |

| | |
|---|---|
| Average CV (%) | 12.7% |

### Parallelism

Parallelism of responses between r14K and endogenous apo(a) was assessed using a plasma with an Lp(a) concentration of 150 nmol/L by ELISA and three sets of samples: 1) standard additions of pure r14K to the plasma sample; 2) serial dilutions of the same plasma sample with “blank” plasma; 3) preparation of the regular calibration curve using r14K and “blank” plasma.

For the standard addition step, the 150 nmol/L Lp(a) plasma sample was gravimetrically supplemented with a 0.5 μmol/L solution of r14K in 100 mmol/L AmBic to achieve 3 concentrations (220, 260 and 300 nmol/L). The standard addition curve, i.e. the linear regression curve of the ratios of non-labelled peptide (endogenous and/or recombinant) to SIL peptide as a function of the concentration, was constructed. Using this curve, the concentration of the sample was back-calculated. In parallel, the same 150 nmol/L plasma was gravimetrically diluted in “blank” plasma to achieve 3 concentrations (40, 80 and 120 nmol/L) and a dilution linear regression curve was plotted. Finally, a calibration curve using r14K was prepared using the same 0.5 μmol/L r14K solution used for the standard addition step. Parallelism of these 3 regression curves was assessed in two independent assays with freshly prepared samples and calibrators by comparing the slopes and their respective 95% confidence intervals (CI).

### Estimation of the Associated Measurement Uncertainties

The estimated measurement uncertainties for the WHO/IFCC SRM-2B reference material were based on the assumption that the uncertainty associated to the measurement of each individual peptide concentration could be estimated based on a type A error. This consisted in calculating the standard deviation (SD) of the measurements divided by the square root of the number of independent replicate measurements (n). WHO/IFCC SRM2B reference was measured by nine independent measurements (n=9). The relative estimate uncertainty associated to each peptide was calculated by dividing this uncertainty by the mean concentration for each individual peptide.

$${u}_{rel Pep} = \frac{SD}{\sqrt{n}} \times \frac{1}{{pep}_{mean ( n = 9 )}}$$

To calculate the relative uncertainty associated with the three-peptide mean, the law of uncertainty propagation was used and the square root of the sum of the squares of each peptide relative uncertainty was calculated, divided by the three-peptide mean:

$${u}_{rel}^{3 pep mean} = \frac{\sqrt{( {u}_{relTPAY}^{2} + {u}_{relTPEN}^{2} + {u}_{relGISS}^{2} )}}{\overline{3 pep}}$$

A first estimate of the three-peptide mean uncertainty was obtained. However, given that results are the average of three individual peptides, a component of fidelity was added to this estimated uncertainty. This component was calculated as the between peptide variability extracted from a one-way ANOVA statistical analysis performed between the measurement series of the three individual peptides. Using again the law of uncertainty propagation and a k=2 enlarging factor, a final estimate of the uncertainty associated with Lp(a) concentration for the WHO/IFCC SRM2B reference material measured by LC-MS/MS was determined.

Of note, this estimate of the uncertainty associated with the measurement of Lp(a) using this LC-MS/MS method is not an accurate uncertainty budget and that a number of uncertainty sources are not taken into account using this model. In particular, the uncertainties associated to the calibration material and to the linearity of the calibration curve are not included in this model.

### Results

### The Recombinant apo(a) as a Primary Calibration Material

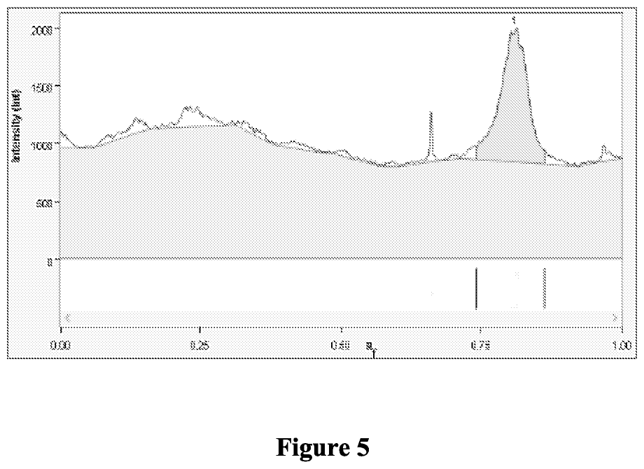

A recombinant apo(a) was selected as calibrator because, unlike proteotypic tryptic peptides, it could control variability introduced by sample handling and enzymatic digestion. Migration of r14K on agarose gel electrophoresis confirmed that the protein contained 14 MV repeats (FIG. 5). Purity of the preparation was assessed by a combination of orthogonal methods that all indicated purity of 95% or better (FIG. 6).

The concentration of r14K was determined at NIST using the amino-acid analysis higher order reference method (Table 6). The two hydrolysis conditions yielded indistinguishable results and were combined to calculate r14K concentration. For each AA quantified, CVs across the replicate measurements were below 1.5% and between-AA CV was 3.8% across all assays. Based on the overall data and using a molecular weight 211,992.4 g/mol, a value of 5.00±0.15 μmol/L was assigned to the r14K as a primary reference material.

Table 6 provides measurement of the concentration of the r14K recombinant apo(a) by amino-acid analysis. Hydrolysis 1, 130° C. for 48 h; hydrolysis 2, 140° C. for 71 h.

TABLE 6

| | Hydrolysis 1 | | Hydrolysis 2 | | Overall | |
|---|---|---|---|---|---|---|
| Amino Acid | Mean (g/L) | % CV | Mean (g/L) | % CV | Mean (g/L) | % CV |
| Phe | 1.10 | 0.72 | 1.11 | 1.20 | 1.11 | 1.00 |
| Leu | 1.11 | 1.80 | 1.06 | 1.70 | 1.09 | 3.20 |
| Ile | 1.14 | 1.90 | 1.06 | 1.50 | 1.10 | 3.90 |
| Val | 1.07 | 0.54 | 1.02 | 0.58 | 1.04 | 2.80 |
| Ala | 0.97 | 0.64 | 0.92 | 0.55 | 0.95 | 2.60 |
| Pro | 1.00 | 0.88 | 0.98 | 0.48 | 0.99 | 1.30 |
| All combined | 1.08 | 5.20 | 1.04 | 5.40 | 1.06 | 5.70 |
| Conc. (μmol/L) | 5.10 | | 4.91 | | 5.00 | |

Phe, Phenylalanine;
Leu, Leucine;
Ile, Isoleucine;
Val, Valine;
Ala, Alanine;
Pro, Proline

### Method Development

In preliminary experiments, six candidate quantification peptides were monitored in tryptic digests of r14K and plasma samples, with at least four selected reaction monitoring transitions for each endogenous and SIL peptide (Table 7).

Table 7 provides candidate peptides identified from data independent analysis of the human recombinant r14K apolipoprotein(a) in LC-MS/MS. For each peptide, short-names, peptide amino-acid sequence, location in the protein and number of repeats are indicated. LC-MS/MS parameters, retention time window, precursor mass and mass over charge ratios, transitions followed, and collision energies used are indicated. MV: Kringle IV

TABLE 7

| Short- | Full peptide | | Copies per | Retention time | Precursor |
|---|---|---|---|---|---|
| name | sequence | Location | apo(a) | window (min) | ion (m/z) |
| TPAY | TPAYYPNAGLIK | KIV-4 | 1 | 16.5-19.5 | 654.3533 |
| | (SEQ ID NO: 2) | | | | |
| TPEN | TPENYPNAGLTR | KIV-5 | 1 | 12.0-15.0 | 666.8308 |
| | (SEQ ID NO: 1) | | | | |
| GSFS | GSFSTTVTGR | KIV-6 | 2 | 11.5-14.5 | 506.7565 |
| | (SEQ ID NO: 5) | & 7 | | | |
| TTEY | TTEYYPNGGLTR | KIV-6 | 2 | 13.5-16.5 | 686.3306 |
| | (SEQ ID NO: 6) | & 7 | | | |
| GISS | GISSTTVTGR | KIV-9 | 1 | 10.0-13.0 | 489.7644 |
| | (SEQ ID NO: 3) | | | | |
| LFLE | LFLEPTQADIALLK | Protease | 1 | 23.0-26.0 | 786.4558 |
| | (SEQ ID NO: 4) | domain | | | |

| Short- | Collision | Ion | Transition | Interference |
|---|---|---|---|---|
| name | energy(V) | type | (m/z) | (endogenous or SIL) |
| TPAY | 22.5 | y10 | 1109.60 | — |
| | | y9 | 1038.56 | — |
| | | y8 | 875.50 | — |
| | | y7 | 712.44 | — |
| TPEN | 28.6 | y10 | 1134.55 | — |
| | | y9 | 1005.51 | Y |
| | | y8 | 891.47 | — |
| | | y7 | 728.41 | — |
| GSFS | 18.1 | y7 | 721.38 | Y |
| | | y6 | 634.35 | — |
| | | y5 | 533.30 | Y |
| | | y4 | 432.26 | Y |
| TTEY | 23.5 | y10 | 1169.56 | Y |
| | | y8 | 877.45 | Y |
| | | y7 | 714.39 | -- |
| GISS | 17.6 | y8 | 808.42 | — |
| | | y7 | 721.38 | — |
| | | y5 | 533.30 | — |
| | | y3 | 333.19 | — |
| LFLE | 26.5 | y11 | 1198.67 | Y |
| | | y10 | 1069.63 | — |

Because the major requirements of the method were accuracy and robustness, stringent criteria were chosen to select the suitable peptides. Three of the six peptides, TTEY, GSFS and LFLE, displayed matrix interferences in all but a single transition for either endogenous or SIL peptide (not meeting CLSI recommendations C62-A) and demonstrated instability under assay conditions (TTEY) or inadequate robustness (GSFS, LFLE) in the intermediate precision study (CV>10% at LLOQ) and were excluded from consideration. Three peptides, TPEN, TPAY and GISS, fulfilled CLSI criteria (C62-A) and were selected for further evaluation (Table 8). Moreover, it was suggested that for peptide-based quantification of proteins in LC-MS/MS, combining the results of multiple peptides improves robustness and accuracy of the assay. The ultimate measure of Lp(a) concentration was therefore the mean response of the three selected peptides.

Table 8 provides characteristics and MS/MS parameters of the three quantification peptides selected for the Lp(a) LC-MS/MS method.

TABLE 8

| | | | Normalized | | |
|---|---|---|---|---|---|
| | Retention time | Precursor | Collision | Ion | Transition |
| Name | window (min) | ion (m/z) | energy (V) | type | (m/z) |
| TPAY | 16.5-19.5 | 654.35 | 22.5 | y10 | 1109.6 |
| | | | | y9 | 1038.6 |
| | | | | y8 | 875.5 |
| | | | | y7 | 712.4 |
| TPEN | 12.0-15.0 | 666.83 | 28.6 | y10 | 1134.6 |
| | | | | y8 | 891.5 |
| | | | | y7 | 728.4 |
| GISS | 10.0-13.0 | 489.76 | 17.6 | y8 | 808.4 |
| | | | | y7 | 721.4 |
| | | | | y5 | 533.3 |
| | | | | y3 | 333.2 |

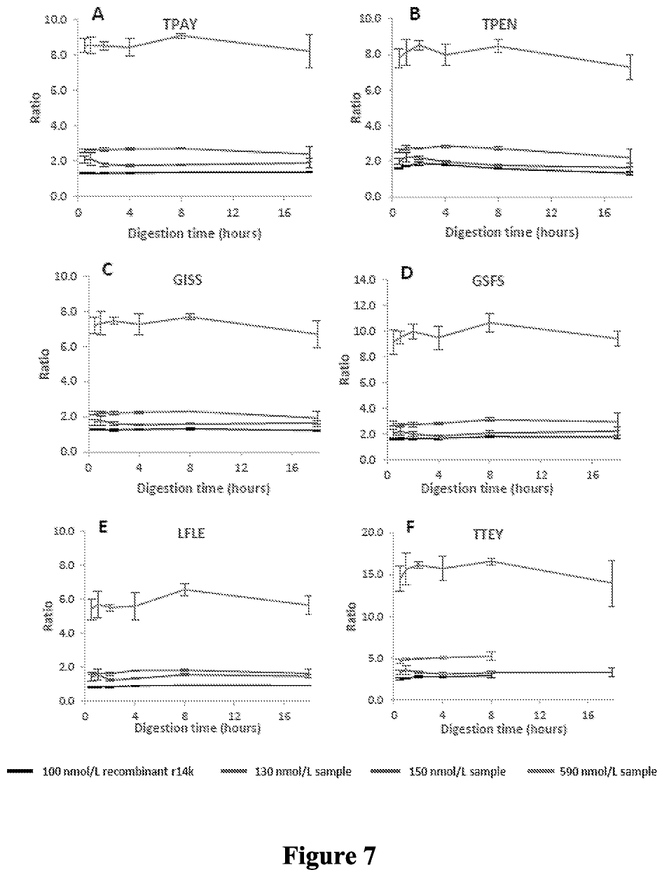

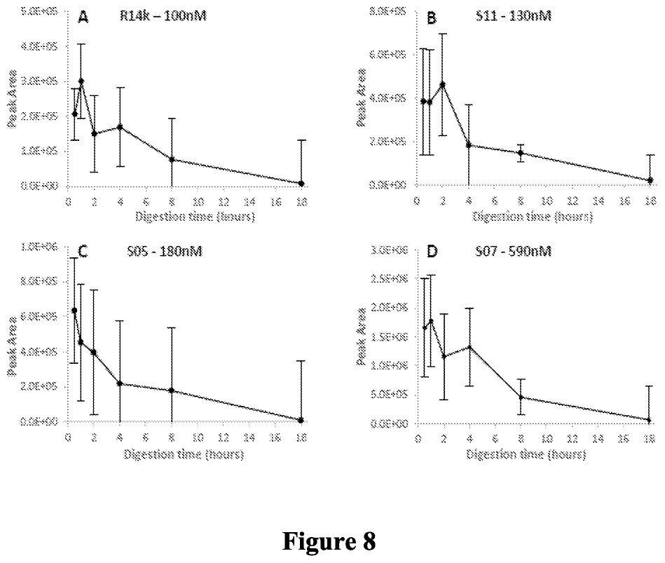

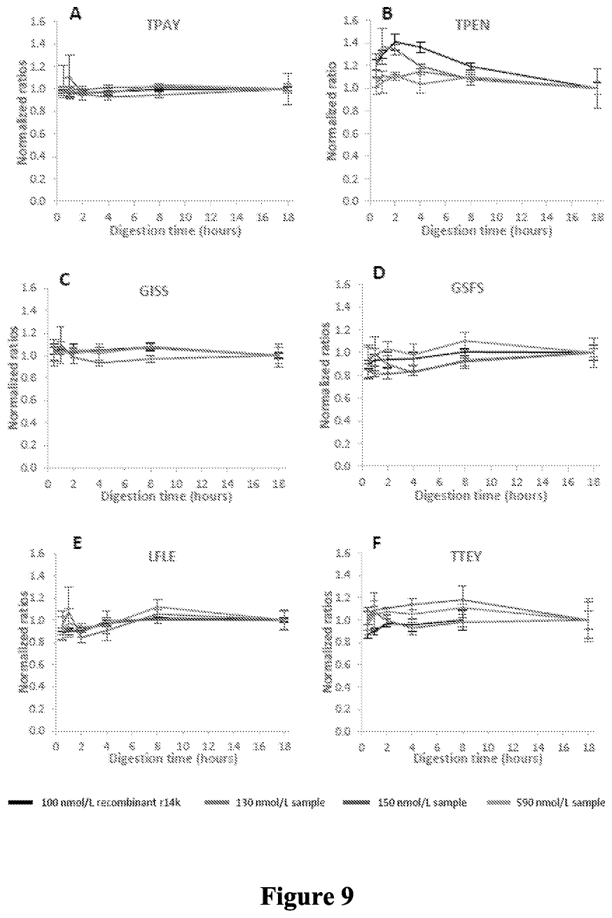

To ensure comparable digestion kinetics and to assess the effect of apo(a) isoform size on digestion, time-course experiments were performed. For the three peptides, the ratio of endogenous to SIL peptide reached maximum at the first time-point (30 min) and the data did not suggest any trend for overall changes (FIGS. 7 and 8). Peptide response across the digestion time-course after normalization to t=18 h overlapped at all time-points independent of apo(a) isoform for all peptides (FIG. 9) and no differences were observed in the digestion time-course between samples, thus confirming that the digestion of Lp(a) was robust and that apo(a) isoform size did not affect digestion kinetics.

### Parallelism

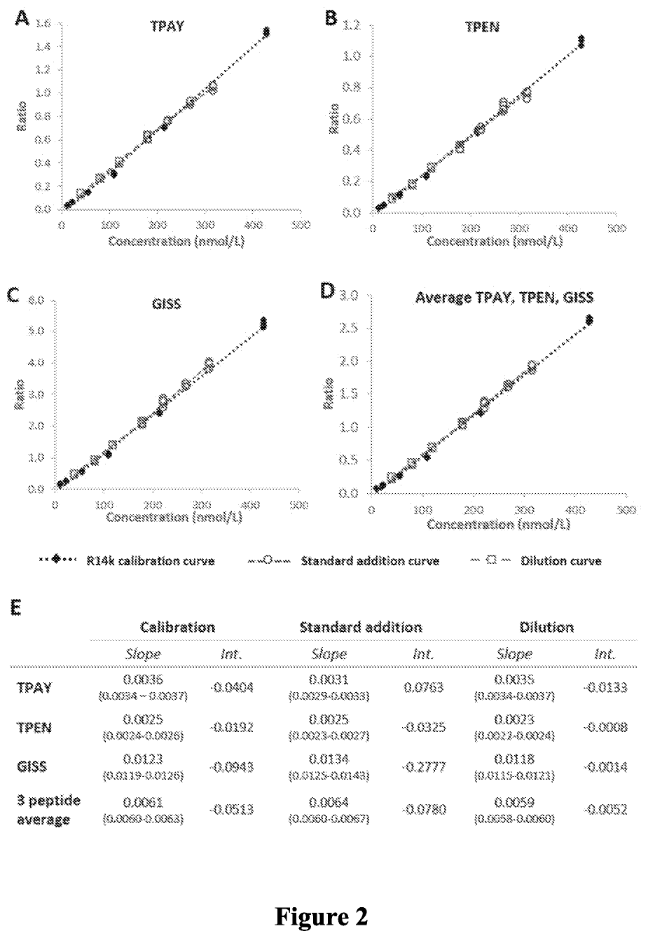

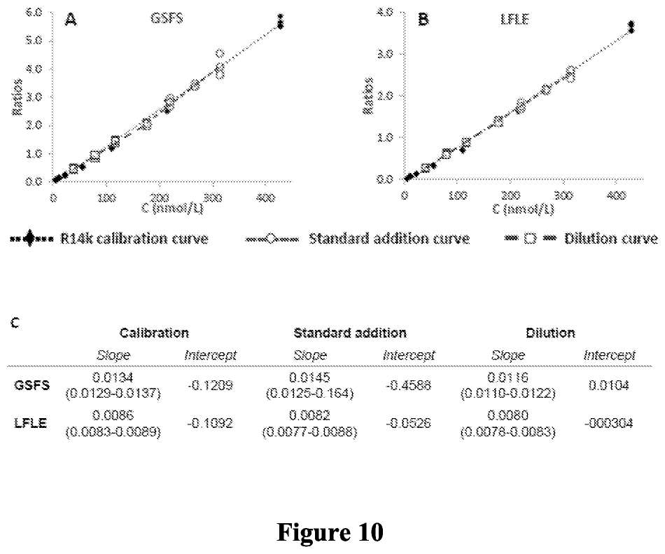

To verify the suitability of r14K as calibrator, parallelism of responses between r14K and endogenous apo(a) was assessed. For TPEN, TPAY and GISS individually, as well as for their mean, linear regressions of the calibration curve (r14K only), the standard additions curve (r14K added to endogenous apo(a)), and the serial dilutions curve (endogenous apo(a) only) provided similar slopes and intercepts (FIG. 2A-D, FIG. 10). The slopes and 95% CI for each individual peptide and their mean were not notably different with overlapping 95% CI (FIG. 2E), confirming the suitability of r14K as calibrator.

### Method Validation

Linearity, LOD and LLOQ for the three final peptides were determined from a serial dilution of r14K in “blank” plasma in three separate assays. Linearity (r²>0.994) was determined from 10 nmol/L up to 400 nmol/L; LOD ranged from 4.9 to 10.4 nmol/L, and LLOQ varied between 10 and 20 nmol/L (Table 9).

Table 9. Linearity (r²), limits of detection (LODs) and lower limits of quantification (LLOQs) for the candidate quantification peptides. Linearity was assessed on 3 independent calibrations. LODs were calculated as “blank” plus 2 standard deviations (SD) in Skyline on “blank” plasma spiked with SIL peptides, digested and injected in duplicate (n=4). LLOQ was calculated according to CLSI EP-17 and based on 15% bias and 20% coefficient of variation (CV) at LLOQ.

TABLE 9

| | |
|---|---|
| Linearity (r²) | Limit of Detection (nmol/L) |

| Peptide | Day 1 | Day 2 | Day 3 | Mean | CV (%) | Day 1 | Day 2 | Day 3 | Mean | CV (%) |
|---|---|---|---|---|---|---|---|---|---|---|
| TPAY | 0.9965 | 0.9944 | 0.9947 | 0.9952 | 0.1% | 5.5 | 7.3 | 9.6 | 7.5 | 27.7% |
| TPEN | 0.9928 | 0.9936 | 0.9959 | 0.9941 | 0.2% | 6.1 | 6.3 | 5.0 | 5.8 | 12.2% |
| GISS | 0.9924 | 0.9930 | 0.9957 | 0.9937 | 0.2% | 5.6 | 8.2 | 6.3 | 6.7 | 19.7% |
| GSFS | 0.9958 | 0.9938 | 0.9929 | 0.9942 | 0.1% | 8.0 | 10.4 | 7.8 | 8.8 | 16.6% |
| LFLE | 0.9906 | 0.9897 | 0.9829 | 0.9877 | 0.4% | 3.5 | 6.1 | 4.9 | 4.9 | 26.5% |

Limit of Quantification (nmol/L)

| Peptide | Day 1 | Day 2 | Day 3 | Mean |
|---|---|---|---|---|
| TPAY | 9.4 | 21.6 | 9.7 | 13.6 |
| TPEN | 19.7 | 21.6 | 9.7 | 17.0 |
| GISS | 19.7 | 21.6 | 9.7 | 17.0 |
| GSFS | 19.7 | 51.2 | 19.4 | 30.1 |
| LFLE | 19.7 | 21.6 | 9.7 | 17.0 |

In the repeatability and intermediate precision assessment (Table 10), the Grubbs test for outliers detected two outliers for TPEN at the lowest concentration. These outliers were removed from the analysis. Technical replicate imprecision at LLOQ (n=3 injections) ranged from 0.2% to 12.7% with a mean of 4.1% (SD±3.2%) (Table 10). For each measured peptide, intra-day imprecision (n=9) was below 8% with a mean of 3.9±1.6%, 4.0±1.5% and 3.9±1.5% respectively (Table 10).

Table 10 shows repeatability and intermediate precision of the three final quantification peptides and the 3-peptide mean. The mean CV per peptide was additionally calculated as an indicator or each peptide performances.

TABLE 10

| | | Intra-day imprecision % CV (n = 9) | | | | Inter-day imprecision % CV (n = 27) | | | |
|---|---|---|---|---|---|---|---|---|---|
| | | | | | 3-peptide | | | | 3-peptide |
| Sample | Day | TPAY | TPEN | GISS | mean | TPAY | TPEN | GISS | mean |
| S01 | 1 | 5.2% | 3.9% | 5.2% | 6.8% | 7.3% | 4.8% | 8.9% | 5.0% |
| | 2 | 6.7% | 6.2% | 7.3% | 7.7% | | | | |
| | 3 | 4.8% | 4.0% | 7.3% | 6.3% | | | | |
| S02 | 1 | 4.6% | 5.5% | 3.5% | 5.2% | 7.1% | 6.0% | 5.2% | 4.1% |
| | 2 | 6.4% | 7.0% | 4.3% | 7.5% | | | | |
| | 3 | 7.4% | 5.7% | 4.7% | 7.3% | | | | |
| S03 | 1 | 2.6% | 2.7% | 2.8% | 5.5% | 3.8% | 4.1% | 5.0% | 3.1% |
| | 2 | 3.2% | 4.8% | 4.0% | 5.9% | | | | |
| | 3 | 3.8% | 5.0% | 3.4% | 5.5% | | | | |
| S05 | 1 | 2.3% | 3.6% | 2.2% | 6.3% | 3.0% | 3.6% | 2.5% | 1.7% |
| | 2 | 2.7% | 3.8% | 2.4% | 7.6% | | | | |
| | 3 | 2.7% | 3.6% | 2.9% | 7.6% | | | | |
| S07 | 1 | 2.8% | 2.0% | 2.5% | 4.6% | 3.7% | 1.8% | 3.5% | 2.2% |
| | 2 | 3.9% | 1.8% | 3.0% | 5.4% | | | | |
| | 3 | 3.4% | 1.8% | 2.9% | 5.0% | | | | |
| S07-Dil | 1 | 3.0% | 3.5% | 4.2% | 5.3% | 3.5% | 3.9% | 4.8% | 2.9% |
| | 2 | 2.4% | 2.3% | 4.0% | 5.4% | | | | |
| | 3 | 2.4% | 4.1% | 3.5% | 5.3% | | | | |

| | | | | | | | | |
|---|---|---|---|---|---|---|---|---|
| Mean CV | 3.9% | 4.0% | 3.9% | 6.1% | 4.7% | 4.0% | 5.0% | 3.2% |

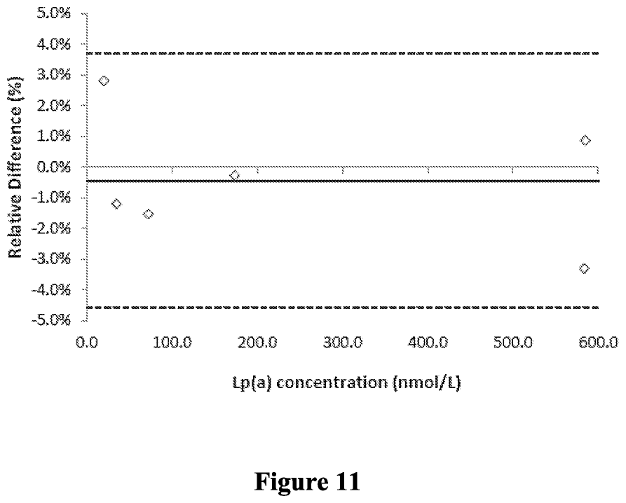

Inter-day imprecision across concentrations did not exceed 8% for TPEN and TPAY and 9% for GISS. The results were not found significantly different for the 590.5 nmol/L sample when measured directly or diluted (two-way ANOVA, α=0.05), with similar CVs for all peptides. For the three-peptide mean, the intra-day imprecisions matched those of individual peptides (<8%). However, the inter-day imprecision was markedly improved over individual peptides with no sample exceeding 5% (mean 3.2±1.2%). In preliminary experiments, the method was evaluated in another laboratory using a high-throughput normal-flow LC-MS/MS. While intra-day and inter-day CVs were higher, the results were very consistent across platforms with inter-laboratory imprecision <10% and mean bias between the two laboratories <2% (FIG. 11, Tables 4 and 5).

### Method Comparison to the ELISA Gold Standard

Comparison of results obtained with the LC-MS/MS method and the current gold standard ELISA was performed on a set of 64 well characterized samples with defined apo(a) isoforms. The secondary reference material WHO/IFCC SRM-2B was additionally evaluated. The value of 104.7±8.4 nmol/L (k=2 uncertainty) determined by LC-MS/MS was in excellent agreement with the assigned value of 107 nmol/L (Table 11).

Table 11 provides results of the measurement of WHO/IFCC SRM-2B reference material for apo(a) by LC MS/MS. Assays correspond to independent calibrations with a fresh aliquot of r14K, digests correspond to a fresh aliquot of SRM-2B. Each digest was injected in duplicate. The 3-peptide mean, standard deviation (SD) and variation coefficient (CV) across each injection were calculated with overall mean per peptide and across assays.

TABLE 11

| | | TPAY | TPEN | GISS | Mean per injection | | |
|---|---|---|---|---|---|---|---|
| Digest | | (nmol/L) | (nmol/L) | (nmol/L) | 3pep | SD | CV(%) |
| Assay 1 | 1 | 104.7 | 103.7 | 112 | 106.8 | 4.5 | 4.2% |
| | | 106.2 | 101.5 | 101.5 | 103.1 | 2.7 | 2.7% |
| | 2 | 106.7 | 102.9 | 99.3 | 102.9 | 3.7 | 3.6% |
| | | 115.8 | 106.2 | 105.8 | 109.3 | 5.6 | 5.2% |
| Assay 2 | 1 | 109.6 | 111.7 | 107.6 | 109.6 | 2.1 | 1.9% |
| | | 110 | 104.6 | 101.2 | 105.3 | 4.5 | 4.2% |
| | 2 | 114.2 | 96.4 | 101.8 | 104.1 | 9.1 | 8.7% |
| | | 112.9 | 110.1 | 103.9 | 109 | 4.6 | 4.2% |
| Assay 3* | 2 | 106 | 97.1 | 101.3 | 101.5 | 4.5 | 4.4% |
| | | 103.7 | 95.7 | 99.7 | 99.7 | 4 | 4.0% |
| Assay 4 | 1 | 107.6 | 102.9 | 94 | 101.5 | 6.9 | 6.8% |
| | | 109.6 | 100.3 | 100.9 | 103.6 | 5.2 | 5.0% |
| | 2 | 108.8 | 97.6 | 97 | 101.1 | 6.7 | 6.6% |
| | | 109 | 100.4 | 101.1 | 103.5 | 4.8 | 4.6% |
| | 3 | 106.7 | 101.5 | 107.6 | 105.3 | 3.3 | 3.1% |
| | | 108.4 | 104.8 | 104.6 | 106 | 2.2 | 2.0% |
| | 4 | 110.7 | 109.5 | 105.4 | 108.5 | 2.8 | 2.6% |
| | | 108.2 | 99 | 103 | 103.4 | 4.6 | 4.5% |

| | | | | |
|---|---|---|---|---|
| Mean | 108.8 | 102.5 | 102.6 | 104.7 |
| SD | 3.2 | 4.7 | 4.2 | 5.0 |
| CV (%) | 2.9% | 4.6% | 4.1% | 4.8% |

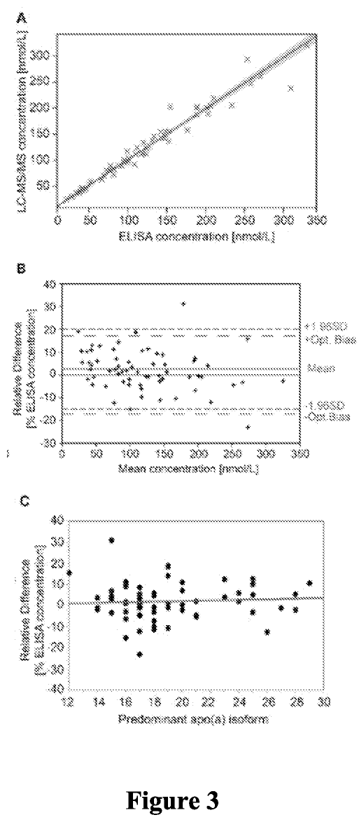

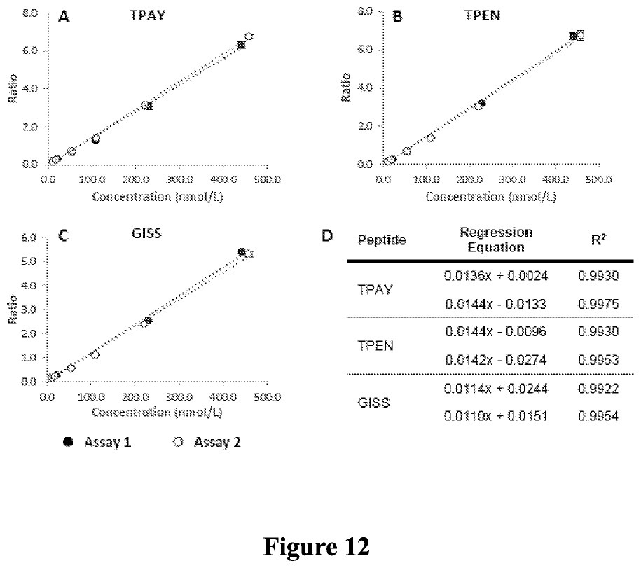

The calibration curves in the two assays comparing LC-MS/MS and ELISA, repeated on different days, were indistinguishable (FIG. 12). Results of the 64 samples showed a Pearson correlation r²=0.958. The weighted-Deming regression analysis demonstrated agreement between the two methods with y=3.18[95% CI:1.08−5.28]+EL/SA*0.98[95% C1:0.94−1.02] (FIG. 3A). The Bland-Altman difference plot (FIG. 3B) indicated minimal differences of LC-MS/MS values compared to ELISA with a 1.7 nmol/L mean difference (2.5%) [1.96×SD limits of agreement −29.8 to 33.2 nmol/L].

The intercept of the Deming regression was in excellent agreement with the low, but not negligible, concentration of Lp(a) in the “blank” plasma (4.9 nmol/L). However, correction for the “blank” plasma contribution to peak area of endogenous peptides did not influence results of this comparison (Table 3). Correlation between the relative bias and the primary apo(a) isoform size expressed in number of KIV (y=0.13×−0.4, r²=0.003, N.S.) indicated that the variation in KIV-2 number accounted for at most 0.3% of the bias variation, confirming that apo(a) size polymorphism did not affect the LC-MS/MS results (FIG. 3C).

In summary, we selected a recombinant apo(a) as the external primary calibrator that was expressed in human HEK293 cells providing glycosylation and folding patterns similar to endogenous protein, a factor considered for the accuracy of the method. We further combined it with SIL peptides as internal standards, an approach that has demonstrated both precision and accuracy. Purity and SI-traceable concentration of the recombinant apo(a) calibrator further ensure traceability of the method, in contrast to the calibrators of previously published LC-MS/MS methods.

Because of its complex, repetitive sequence with a large number of homologous peptides throughout the MV domains and the variable number of MV-2 repeats, apo(a) is a major challenge for LC-MS/MS. Previously published methods selected different tryptic peptides, and in particular a peptide LFLEPTQADIALLK (SEQ ID NO: 4) found in the protease-like domain of apo(a). However, upon extensive validation on nano- and normal-flow UPLC-MS/MS, this peptide demonstrated lower precision. We therefore selected three other peptides with better accuracy, precision, and robustness.

Using these peptides, we demonstrated excellent parallelism between the recombinant apo(a) calibrator and endogenous Lp(a)-associated apo(a), a key parameter for accuracy. Our LC-MS/MS method accurately measured the concentration of the WHO/IFCC SRM-2B reference material for Lp(a) that was value-assigned using isolated Lp(a) and amino-acid analysis. Similarly, the results obtained by the LC-MS/MS method on 64 clinical samples were in excellent agreement with those obtained by the current gold standard ELISA. We additionally demonstrated that, like this ELISA assay, results are not affected by the size polymorphism of apo(a).

Finally, one particular requirement defined by the JCTLM for reference methods is the definition of measurement uncertainties that are fit-for-purpose. It is commonly accepted that the uncertainties associated to a reference method should be a third of the total allowable error defined for assays. For Lp(a), defining total allowable error is challenging because of the lack of sound biological variability data to properly define analytical performance specifications. Nevertheless, current guidelines from the European Federation of Clinical Chemistry and Laboratory Medicine recommend a desirable 40.3% and an optimum 20.1% total allowable error for Lp(a) assays. Based on these guidelines, a suitable reference method should have uncertainties below 12%, or optimally below 7%. The 8.0% relative uncertainty estimated for the WHO/IFCC SRM-2B using the LC-MS/MS method of the disclosure is in-line with current recommendations.

Altogether, results demonstrate the suitability of recombinant apo(a) as primary reference material for the quantification of Lp(a) in plasma by LC-MS/MS. The method of the disclosure additionally fulfills quality requirements to be proposed as a candidate reference method for the standardization of Lp(a) assays. A clearly defined measurand, i.e., the quantity intended to be measured, is needed to set a new traceability chain. The choice of a full-length protein as primary calibrator, while it has notable advantages for accuracy and robustness, represents a challenge for the definition of the measurand because what constitutes the calibrator and the quantity measured, i.e., proteotypic peptides, differ. In certain embodiments, the mean of three peptides distributed across apo(a) sequence may be used as a surrogate for the full-length apo(a). To ensure the high quality of the method and to confirm the validity of this choice, an extensive validation was performed and found that the use of the three-peptide mean was robust across samples and demonstrated comparability to the current designated comparison method.

The method of the disclosure is directly traceable to SI units, independent of Lp(a) isoform size and demonstrates precision, robustness and accuracy. These characteristics, coupled with the excellent comparability of results with the current gold standard ELISA, underscore its potential for the standardization of Lp(a) assays.

### Definitions

Unless specifically defined herein, all terms used herein have the same meaning as they would to one skilled in the art of the present disclosure.

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood other embodiments may include more or less of each element shown in a given Figure. Further, some of the illustrated elements may be combined or omitted. Yet further, an example embodiment may include elements not illustrated in the Figures. As used herein, with respect to measurements, “about” means +/−5%.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

It is understood that the examples and embodiments described herein are for illustrative purposes only and that various modifications or changes in light thereof will be suggested to persons skilled in the art and are to be incorporated within the spirit and purview of this application and scope of the appended claims.

1. A method for quantifying apolipoprotein(a) in a biological sample, the method comprising:
measuring an ion signal of at least two peptides selected from TPENYPNAGLTR (SEQ ID NO: 1) (TPEN), TPAYYPNAGLIK (SEQ ID NO: 2) (TPAY), and GISSTTVTGR (SEQ ID NO: 3) (GISS) and an ion signal of their respective stable isotope labeled peptides in the biological sample by mass spectrometry analysis;
normalizing the ion signal of at least two candidate peptides with the ion signal of stable isotope labeled peptides to obtain an average ion signal of the at least two peptides; and
quantifying apolipoprotein(a) using the average ion signal of the at least two peptides and a reference amount of a calibrator.

2. (canceled)

3. The method of claim 1, the ion signal of at least three peptides selected from TPEN, TPAY, and GISS is measured.

4. The method of claim 1, wherein LFLEPTQADIALLK (SEQ ID NO: 4) (LFLE) and/or GSFSTTVTGR (SEQ ID NO: 5) (GSFS) is measured.

5. The method of claim 1, further comprising first treating the biological sample with a protease that digests the apolipoprotein(a) into at least two peptides selected from TPEN, TPAY, and GISS.

6. (canceled)

7. The method of claim 1, further comprising adding an amount of the respective stable isotope labeled peptides to the biological sample prior to measuring.

8. The method of claim 1, wherein the ion signal of the peptides in the biological sample relative to the amount of two of more internal standards and to a reference amount of a calibrator is indicative of an amount of apolipoprotein(a) in the biological sample.

9. The method of claim 1, wherein the two or more internal standards comprises the respective stable isotope labeled peptides.

10. The method of claim 1, wherein the calibrator comprises a calibrated reference sample selected from calibrated plasma sample (such as single or pooled), serum sample (such as single or pooled), and plasma or serum lacking Lp(a) spiked with a known amount of purified Lp(a) or 14K recombinant apo(a).

11. The method of claim 10, wherein the calibrator is obtained by calibrating the a reference sample with a primary reference material, the calibration comprising:
measuring an ion signal of at least two peptides selected from TPEN, TPAY, and GISS in the reference sample; and
assigning the reference ion signal of the peptides based on the amount of the primary reference material and one of more internal standards comprising stable isotope labeled peptides to be measured.

12. The method of claim 11, wherein the primary reference material comprises a high-purity human recombinant apolipoprotein(a).

13. (canceled)

14. A method for determining if a subject is at risk to develop a cardiovascular disease, the method comprising:
quantifying the amount of apolipoprotein(a) in a biological sample according to the method of claim 1, and
comparing the amount of apolipoprotein(a) to a predetermined amount of lipoprotein(a), wherein a difference in the amount of apolipoprotein(a) in the biological sample and the predetermined amount of lipoprotein(a) is indicative of the risk to develop cardiovascular disease in the subject.

15. The method of claim 14, wherein an increased amount of apolipoprotein(a) in the biological sample compared to the predetermined amount is indicative of the risk of developing cardiovascular disease in the subject.

16. The method of claim 14, wherein the cardiovascular disease is myocardial infarction, atherosclerosis, coronary artery disease, peripheral artery disease, heart failure, stroke, arterial thrombosis, calcific aortic valve disease, aortic stenosis, or venous thromboembolism.

17. (canceled)

18. (canceled)

19. (canceled)

20. (canceled)

21. (canceled)

22. (canceled)

23. (canceled)

24. (canceled)

25. The method of claim 1, wherein the biological sample is a blood sample, a serum sample, or a plasma sample.

26. (canceled)

27. (canceled)

28. A method of determining a reference average ion signal of at least two peptides in a reference sample, the method comprising:
calibrating the reference sample with a primary reference material comprising the high-purity human recombinant apolipoprotein(a);
identifying two or more peptides for quantification based on a group of factors to obtain the two or more peptides; and
determining the reference average ion signal of the two or more peptides normalized to ion signal of their respective stable isotope labeled peptides based on the amount of the primary reference material.

29. The method of claim 28, wherein the high-purity human recombinant apolipoprotein(a) is r14K (SEQ ID NO: 7).

30. The method of claim 28 or claim 29, wherein the group of factors to obtain the two or more peptides comprise: a level of presence of the high-purity recombinant apolipoprotein(a) in a KIV-2 domain; a level of absence of an amino acid susceptible to a modification; a level of absence of homologous peptides in a human proteome; a level of absence of known human genetic mutations; and a combination thereof

31. The method of claim 28, wherein the reference sample is selected from plasma sample (such as single or pooled), serum sample (such as single or pooled), and plasma or serum lacking Lp(a) and spiked with a known amount of purified Lp(a) or 14K recombinant apo(a).

32. (canceled)

33. A kit comprising: (i) two or more stable isotope labeled peptides selected from TPEN (SEQ ID NO: 1), TPAY (SEQ ID NO: 2), and GISS (SEQ ID NO: 3), and optionally (ii) one or more of calibrators.

34. (canceled)

35. (canceled)

36. (canceled)

37. (canceled)

38. (canceled)

39. (canceled)

40. (canceled)

41. (canceled)

42. A non-transitory computer-readable medium having computer-executable instructions stored thereon that, if executed by one or more processors of a computing device, cause the computing device to perform the normalizing of an ion signal of two or more peptides as described in claim 1.


SEQUENCE LISTING (11 sequences)

SEQ ID NO: 1 - protein, 12 aa, Artificial Sequence
  Candidate peptide TPEN
TPENYPNAGLTR

SEQ ID NO: 2 - protein, 12 aa, Artificial Sequence
  Candidate peptide TPAY
TPAYYPNAGLIK

SEQ ID NO: 3 - protein, 10 aa, Artificial Sequence
  Candidate peptide GISS
GISSTTVTGR

SEQ ID NO: 4 - protein, 14 aa, Artificial Sequence
  Candidate peptide LFLE
LFLEPTQADIALLK

SEQ ID NO: 5 - protein, 10 aa, Artificial Sequence
  Candidate peptide GSFS
GSFSTTVTGR

SEQ ID NO: 6 - protein, 12 aa, Artificial Sequence
  apo(a) fragment TTEY
TTEYYPNGGLTR

SEQ ID NO: 7 - protein, 1905 aa, Homo sapiens
EQSHVVQDCYHGDGQSYRGTYSTTVTGRTCQAWSSMTPHQHNRTTENYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDPVAAPYCYTRDPSVRWEYCNLTQCSDAEGTAVAPPTITPIPSLEAPSEQAPTEQRPGVQECYHGNGQSYQGTYFITVTGRTCQAWSSMTPHSHSRTPAYYPNAGLIKNYCRNPDPVAAPWCYTTDPSVRWEYCNLTRCSDAEWTAFVPPNVILAPSLEAFFEQALTEETPGVQDCYYHYGQSYRGTYSTTVTGRTCQAWSSMTPHQHSRTPENYPNAGLTRNYCRNPDAEIRPWCYTMDPSVRWEYCNLTQCLVTESSVLATLTVVPDPSTEASSEEAPTEQSPGVQDCYHGDGQSYRGSFSTTVTGRTCQSWSSMTPHWHQRTTEYYPNGGLTRNYCRNPDAEISPWCYTMDPNVRWEYCNLTQCPVTESSVLATSTAVSEQAPTEQSPTVQDCYHGDGQSYRGSFSTTVTGRTCQSWSSMTPHWHQRTTEYYPNGGLTRNYCRNPDAEIRPWCYTMDPSVRWEYCNLTQCPVMESTLLTTPTVVPVPSTELPSEEAPTENSTGVQDCYRGDGQSYRGTLSTTITGRTCQSWSSMTPHWHRRIPLYYPNAGLTRNYCRNPDAEIRPWCYTMDPSVRWEYCNLTRCPVTESSVLTTPTVAPVPSTEAPSEQAPPEKSPVVQDCYHGDGRSYRGISSTTVTGRTCQSWSSMIPHWHQRTPENYPNAGLTENYCRNPDSGKQPWCYTTDPCVRWEYCNLTQCSETESGVLETPTVVPVPSMEAHSEAAPTEQTPVVRQCYHGNGQSYRGTFSTTVTGRTCQSWSSMTPHRHQRTPENYPNDGLTMNYCRNPDADTGPWCFTMDPSIRWEYCNLTRCSDTEGTVVAPPTVIQVPSLGPPSEQDCMFGNGKGYRGKKATTVTGTPCQEWAAQEPHRHSTFIPGTNKWAGLEKNYCRNPDGDINGPWCYTMNPRKLFDYCDIPLCASSSFDCGKPQVEPKKCPGSIVGGCVAHPHSWPWQVSLRTRFGKHFCGGTLISPEWVLTAAHCLKKSSRPSSYKVILGAHQEVNLESHVQEIEVSRLFLEPTQADIALLKLSRPAVITDKVMPACLPSPDYMVTARTECYITGWGETQGTFGTGLLKEAQLLVIENEVCNHYKYICAEHLARGTDSCQGDSGGPLVCFEKDKYILQGVTSWGLGCARPNKPGVYARVSRFVTWIEGMMR

SEQ ID NO: 8 - protein, 14 aa, Artificial Sequence
  isotope heavy peptide LFLE
  MISC_FEATURE (14)..(14): heavy isotope at 13C6 and 15N2
LFLEPTQADIALLK

SEQ ID NO: 9 - protein, 10 aa, Artificial Sequence
  isotope heavy peptide GISS
  MISC_FEATURE (10)..(10): heavy isotope at 13C6 and 15N4
GISSTTVTGR

SEQ ID NO: 10 - protein, 12 aa, Artificial Sequence
  isotope heavy peptide TPAY
  MISC_FEATURE (12)..(12): heavy isotope at 13C6 and 15N2
TPAYYPNAGLIK

SEQ ID NO: 11 - protein, 12 aa, Artificial Sequence
  isotope heavy peptide TPEN
  MISC_FEATURE (12)..(12): heavy isotope at 13C6 and 15N4
TPENYPNAGLTR